(19) United States
(12) Patent Application Publication
(10) Pub. No.: US 2013/0251783 A1
(43) Pub. Date: Sep. 26, 2013

(54) LIPOSOMES CONTAINING PERMEATION ENHANCERS FOR ORAL DRUG DELIVERY

(71) Applicants: Johannes Parmentier, Singapore (SG); Gert Fricker, Dossenheim (DE); Felix Gropp, Munchen (DE); Klaus Hartmann, Heidelberg (DE)

(72) Inventors: Johannes Parmentier, Singapore (SG); Gert Fricker, Dossenheim (DE); Felix Gropp, Munchen (DE); Klaus Hartmann, Heidelberg (DE)

(73) Assignees: Universitat Heidelberg, Heidelberg (DE); MULLER-BORE & PARTNER, Munchen (DE)

(21) Appl. No.: 13/616,064

(22) Filed: Sep. 14, 2012

Related U.S. Application Data
(60) Provisional application No. 61/534,528, filed on Sep. 14, 2011.
(60) Provisional application No. 61/568,319, filed on Dec. 8, 2011.

(51) Int. Cl. A61K 9/127

(52) U.S. Cl. CPC A61K 9/127
USPC 424/450

(57) ABSTRACT

The present invention relates to liposomal compositions and their application for delivery of pharmaceuticals for the treatment of disease.

### FIELD OF THE INVENTION

Disclosed herein are new liposomal compositions and their application for delivery of pharmaceuticals for the treatment of disease.

### BACKGROUND OF THE INVENTION

Oral application is by far the most convenient route for drug delivery, especially for long and repeated therapeutic use. But the development of formulations for the oral administration of BCS Class III drugs, especially macromolecules like proteins, heparin, or oligonucleotides is rendered more difficult for several reasons. Macromolecules are mostly poorly absorbed due to their high molecular weight and hydrophilicity according to Lipinski's rule of five, and peptides may be degraded presystemically in the gastrointestinal tract (GIT) leading to a reduced fraction reaching the intestinal wall [1-3]. This usually results in a bioavailability of less than 1% [4]. In order to overcome these problems, several approaches have been taken, for instance the use of absorption enhancers like surfactants and small molecule carriers, enzyme inhibitors and the use of particulate systems, mostly nanoparticles or liposomes [5-7].

The first approaches to use liposomes for oral delivery were not very encouraging mostly due to poor reproducibility of the results [8, 9]. Nevertheless, liposomes have some important advantages over other delivery systems as they are well characterised and have good biocompatibility and high versatility [10]. It seems reasonable to combine liposomes with other mechanisms for permeation enhancement to further improve absorption of peptides, as it is possible to deliver protein drugs and enhancers together in one vehicle to the enterocytes. This would allow a reduction in the amount of permeation enhancers used and a decrease in toxic side effects. Unfortunately, permeation enhancers are often surfactants, which can interact easily with the liposomal membrane and are known to destabilise or even destroy phospholipid vesicles. But this effect is clearly dependent on the lipid composition and the type and concentration of the enhancers used, allowing theoretically the formation of stable liposomes containing surfactants.

Soon after their discovery in 1965, liposomes were explored for the oral delivery of peptide and protein drugs [8, 9, 22, 65, 66]. Common features of many proteins are their high molecular weight, hydrophilicity and susceptibility to degradation by proteases or low pH leading to a low oral bioavailability [2]. Liposomes might help to stabilise proteins in the gastro-intestinal tract (GIT) and to improve their permeation through the intestinal mucosa. However, also liposomes show instabilities after oral application, especially against not only bile salts but also pancreatic enzymes and the acidic conditions in the stomach [67, 12, 68]. This leads not only to a reduction of intact liposomes reaching the intestinal mucosa but also to a strong leakage of liposomally encapsulated drugs into the GIT, where they are exposed to low pH or proteases. Several approaches were made to improve the stability of liposomes against the harsh conditions in the GIT.

Vesicles can be coated with polymers such as chitosan, polyethylene glycol or pectin not only to improve the membrane integrity but also to provide a mucoadhesivity and to prolong the retention of the formulations in the gut [69-72]. Liposomes made with phospholipids with a glass transition above body temperature or containing other stabilising lipids like gangliosides can survive the gastro-intestinal tract [73-75]. Since their first description by Langworthy in 1977 naturally derived tetraether lipids (TELs) were used in liposomes to improve their properties for vaccine delivery [76-80]. TELs are present in a great variety in both archaeal and bacterial membranes [81, 82]. Their unique properties make them good candidates for the use in liposomes for oral drug delivery. They are less susceptible to hydrolysis and oxidation than normal phospholipids. Furthermore, TELs are membrane spanning and thus can stabilise bilayer membranes. Despite their rigid structure, they have a low glass transition temperature below 0° C. and are therefore easy to handle at room temperature compared to stabilising phospholipids like disteaorylphosphatidylcholine [83-85]. Commonly, the so called archaeosomes are prepared by the polar lipid fraction obtained from archaea and contain a mixture of bipolar TELs.

Even when intact liposomes reach the intestinal mucosa, uptake of encapsulated protein drugs or vesicles is usually very low [93-96]. Protection of the encapsulated drug by use of stabilised liposomes might not be sufficient for most protein drugs to achieve a reasonable bioavailability. Previous attempts to increase the uptake of liposomal carriers and their encapsulated drugs were among others the use of M-cell targeted liposomes or mucoadhesive vesicles [97, 98]. Due to their high versatility concerning composition, liposomes are suitable drug carriers for the use of bio-enhancers. Chemically defined enhancers represent a cost effective way of bioavailability improvement and have already been investigated in the literature [99]. The simultaneous delivery of enhancer and drug in one vehicle could allow a reduction of enhancer needed and thus also a reduction of possible toxic side effects. Unfortunately, most enhancers are surfactants and can destabilise liposomes making a better understanding of their influence on the stability of liposomes for peroral delivery desirable.

To assure a sufficient protection of encapsulated protein, liposomes should maintain their vesicular form and exhibit no leakage of the protein. Furthermore, the influx of small molecules through the lipid bilayer should be minimal to avoid protein denaturation.

D-α-tocopheryl polyethylene glycol 1000 succinate (TPGS 1000) is a nonionic surfactant and was originally used as a water-soluble vitamin E derivative [11]. TPGS 400 just differs in the length of the PEG chain and thus in the hydrophilic/lipophilic balance (HLB). Besides the surfactant characteristics of TPGS, which mostly implies permeation enhancing properties, the PEG chain can contribute to the stabilisation of the liposomes and is known to have a certain mucoadhesivity [12]. Further, TPGS is a known inhibitor of P-glycoprotein [60]. The anionic bile salt derivative cholylsarcosine (CS), the sarcosine (N-methylglycine) conjugate of cholic acid, behaves very similarly to natural occurring conjugated bile acids [13]. This compound has originally been developed for bile acid replacement in patients with a malabsorption syndrome [14, 15]. Bile salts have been in use for a long time as permeation enhancers and there have already been studies with liposomal bile salt formulations [16, 17]. One advantage compared to taurocholic acid, which has previously been used as an excipient, is lack of tumorgenicity of CS. Taurocholic acid may be metabolised by deconjugation and 7-dehydroxylation to deoxycholic acid, which has been reported to have a certain tumorigenic potential [87-90]. However, due to methylation of the amide bond, CS cannot be deconjugated to form deoxycholic acid. In addition, this bile acid derivative has already been tested in humans with short bowel syndrome in the context of bile acid replacement therapies [46, 91]. Cetylpyridinium chloride (CpCl) is a cationic surfactant mostly used as disinfectant but also with applications as permeation enhancer [18]. Furthermore, it can be incorporated into liposomal membranes [19]. Stearylamine (SA) is a cationic lipid and in contrast to the other enhancers used has just little surfactant properties. Incorporated in liposomal membranes, it leads to a positive surface charge and can therefore promote the cellular uptake of the particles [7]. Octadecanethiol (OT) has no surface active properties, but the thiol group shows a certain mucoadhesivity, a principle already successfully used for bioavailability improvement [92].

In summary, a strong need exists to provide novel liposomal compositions for the delivery of protein drugs together with permeation enhancers. Further, a strong need exists to provide novel liposomal compositions having improved stability in the GIT.

This need is satisfied by providing the embodiments characterized in the claims.

### SUMMARY OF THE INVENTION

The ability of various enhancers to form liposomes with the host lipids egg phosphatidylcholine (EPC) and cholesterol was examined and the possible use of the liposomes for oral drug delivery was assessed by investigating their toxicity and the permeation improvement of a dextran derivative in the Caco-2 Transwell® model. FITC-dextran 70 kDa used in this study is a stable macromolecule, which is not a known substrate of any cellular transport mechanism and shows only low interactions with liposomal lipids [20, 21]. Thus, it allows an investigation into the enhancement potential of the liposomal systems with little influence of possible drug/drug carrier interactions.

As disclosed herein, several bio-enhancers were used in liposomes to improve the permeation of dextran through a Caco-2 cell layer. It was possible to form liposomes in good quality with all the tested enhancers. The cytotoxicity of the surfactants differed with their properties like charge and CMC but was always reduced in a liposomal formulation. In the Transwell® model, the formulations with 5% TPGS 400, 10% CS and 2.5% SA and 10% CpCl had an enhancing effect without influencing the TER or the C_{Cl} suggesting a good safety profile.

In the present invention, we use only one single structure, the naturally derived glycerylcaldityl tetraether (GCTE), for the stabilisation of liposomes. GCTE can be obtained after hydrolysis of the polar lipid fraction of Sulfolobus acidocaldarius, followed by several purification steps [86]. The use of a single chemical entity allows a more target-oriented change of liposomal properties and an easier adjustment of their stability in the intestine. Furthermore, a single structure has lower demands on analytical methods and leads to a higher batch to batch consistency in an industrial production process.

Further, in the present invention, we tested the stability of egg phosphatidylcholine (EPC) and cholesterol (Chol) based liposomes with and without GCTE and the bio-enhancers D-α-tocopheryl polyethylene glycol 1000 succinate (TPGS), cholylsarcosine (CS) and octadecanethiol (OT). Furthermore, we tested the liposomal formulations for their stability under acidic conditions, in bile salts and in pancreatin. Change in size and size distribution was monitored to conclude on the vesicular shape of the particles. In addition, leakage of both FITC-dextran (70 kDa) and carboxyfluorescein (CF) was examined; the first as model for a large hydrophilic molecule and the latter to investigate the membrane permeability of small hydrophilic molecules.

Liposomes containing both the stabilising tetraether lipid GCTE and bio-enhancers could be a versatile tool for oral delivery of proteins or other drug substances, which have a low oral bioavailability due to gastro-intestinal degradation and low permeation. In the present application, we could show that GCTE can improve stability of liposomes against sodium taurocholate and especially could reduce the destabilising effect of bio-enhancers in the liposomal membrane.

### DETAILED DESCRIPTION OF THE INVENTION

The Caco-2 model is the most commonly used in vitro model for oral delivery studies but it has some serious disadvantages like the lack of caveolae or the missing of intestinal fluids and a mucus layer [63,64]. In addition, the liposomes should not just enhance the permeation of proteins but also protect them from the harsh conditions in the intestine. It can be hypothesised that the liposomal formulations would give a better performance in comparison with a free protein in an in vivo model, where the stabilising effect and the interaction with the mucus layer are more important compared to the cell model.

Novel pharmaceutical compositions, certain of which have been found to enhance cell permeation and/or drug bioavailability have been discovered, together with methods of synthesizing and using the compounds including methods for the treatment of diseases in a patient by administering the compounds.

In certain embodiments of the present invention, liposomal compositions as disclosed herein have useful drug bioavailability enhancement or cell permeation properties, and may be useful as pharmaceutical compositions for the treatment of diseases. Thus, in broad aspect, certain embodiments also provide pharmaceutical compositions comprising one or more liposomal compositions disclosed herein together with a pharmaceutically active substance, as well as methods of making and using the compounds and compositions. Certain embodiments provide methods for enhancing the bioavailability of pharmaceutically active substances. Other embodiments provide methods for treating a disorder in a patient in need of such treatment, comprising administering to said patient a therapeutically effective amount of a composition according to the present invention. Also provided is the use of certain compositions disclosed herein for use in the manufacture of a medicament for the treatment of a disease.

In certain embodiments, disclosed herein is a liposomal composition comprising a phospholipid; cholesterol; a permeability enhancer; and an active pharmaceutical ingredient.

In further embodiments, said phospholipid is egg phosphatidylcholine.

In further embodiments, said permeability enhancer is selected from the group consisting of D-α-tocopheryl polyethylene glycol succinate, cholylsarcosine, cetylpyridinium chloride, and stearylamine.

In further embodiments, said permeability enhancer is D-α-tocopheryl polyethylene glycol 1000 succinate.

In further embodiments, said permeability enhancer is D-α-tocopheryl polyethylene glycol 400 succinate.

In further embodiments, said permeability enhancer is cholylsarcosine.

In further embodiments, said permeability enhancer is cetylpyridinium chloride.

In further embodiments, said permeability enhancer is stearylamine.

In further embodiments, said composition comprises an aqueous solution of said active pharmaceutical ingredient and a liposome-forming mixture comprising about 40% to about 60% of said phospholipid, preferably about 50% of said phospholipid; about 1% to about 30% of said permeation enhancer; and about 10% to about 59% of said cholesterol.

In further embodiments, said composition comprises an aqueous solution of said active pharmaceutical ingredient and a liposome-forming mixture comprising about 50% of egg phosphatidylcholine; about 5% to about 25% of said permeation enhancer; and about 25% to about 45% of said cholesterol.

In further embodiments, said composition comprises an aqueous solution of said active pharmaceutical ingredient and a liposome-forming mixture comprising 50% of egg phosphatidylcholine; 5% of D-α-tocopheryl polyethylene glycol 1000 succinate; and 45% of cholesterol.

In further embodiments, said composition comprises an aqueous solution of said active pharmaceutical ingredient and a liposome-forming mixture comprising 50% of egg phosphatidylcholine; 5% of D-α-tocopheryl polyethylene glycol 400 succinate; and 45% of cholesterol.

In further embodiments, said composition comprises an aqueous solution of said active pharmaceutical ingredient and a liposome-forming mixture comprising 50% of egg phosphatidylcholine; 10% of cholylsarcosine; and 40% of cholesterol.

In further embodiments, said composition comprises an aqueous solution of said active pharmaceutical ingredient and a liposome-forming mixture comprising 50% of egg phosphatidylcholine; 10% of cholylsarcosine; 2.5% of stearylamine; and 37.5% of cholesterol.

In further embodiments, said composition comprises an aqueous solution of said active pharmaceutical ingredient and a liposome-forming mixture comprising 50% of egg phosphatidylcholine; 10% of stearylamine; and 40% of cholesterol.

In further embodiments, said composition comprises an aqueous solution of said active pharmaceutical ingredient and a liposome-forming mixture comprising 50% of egg phosphatidylcholine; 10% of cetylpyridinium chloride; and 40% of cholesterol.

In further embodiments, said composition comprises an aqueous solution of said active pharmaceutical ingredient and a liposome-forming mixture comprising 50% of egg phosphatidylcholine; 25% of cetylpyridinium chloride; and 25% of cholesterol.

In further embodiments, said liposomes have an average diameter of 100 to 200 nm.

In further embodiments, said liposomes have a polydispersity index of 0.05 to 0.20.

In further embodiments, disclosed herein is a method of treatment of a disease comprising the administration of a therapeutically effective amount of a composition as recited in claim 1 to a patient in need thereof.

In further embodiments, disclosed herein is a method of treatment of a disease comprising the administration of a therapeutically effective amount of a composition as recited in claim 10 to a patient in need thereof.

In further embodiments, disclosed herein is a method of enhancing the permeation of an active pharmaceutical ingredient comprising the administration of a composition as recited in claim 1 to a patient.

In further embodiments, disclosed herein is a method of enhancing the permeation of an active pharmaceutical ingredient comprising the administration of a composition as recited in claim 10 to a patient.

In further embodiments, said permeation is increased by greater than 3-fold.

In certain embodiments, disclosed herein is a liposomal composition comprising a phospholipid; cholesterol; a purified glycerylcaldityl tetraether; a permeability enhancer; and an active pharmaceutical ingredient.

In further embodiments, said phospholipid is egg phosphatidylcholine.

In further embodiments, said permeability enhancer is selected from the group consisting of cholylsarcosine, octadecanethiol, and D-α-tocopheryl polyethylene glycol 1000 succinate.

In further embodiments, the liposomal composition does not contain any further tetraether lipid. In these embodiments, said composition contains only one purified glycerylcaldityl tetraether.

In further embodiments, the liposomal composition comprises an aqueous solution of said active pharmaceutical ingredient and a liposome-forming mixture comprising about 25% to about 80% of said phospholipid, preferably about 50% of said phospholipid; up to about 60% of said cholesterol; about 5% to about 30% of said purified glycerylcaldityl tetraether; and about 1% to about 35% of said permeability enhancers.

In further embodiments, the liposomal composition comprises an aqueous solution of said active pharmaceutical ingredient and a liposome-forming mixture comprising about 36% of said phospholipid; about 40% to about 54% of said cholesterol; about 9% of said purified glycerylcaldityl tetraether; and about 1% to about 15% of said permeability enhancers.

In further embodiments, the liposomal composition comprises an aqueous solution of said active pharmaceutical ingredient and a liposome-forming mixture comprising about 25% to about 55% of egg phosphatidylcholine; about 20% to about 60% of cholesterol; about 5% to about 15% of purified glycerylcaldityl tetraether; and about 1% to about 20% of a permeability enhancer, selected from the group consisting of cholylsarcosine, octadecanethiol, and D-α-tocopheryl polyethylene glycol 1000 succinate.

In further embodiments, the liposomal composition comprises an aqueous solution of said active pharmaceutical ingredient and a liposome-forming mixture comprising about 36% of egg phosphatidylcholine; about 40% to about 54% of cholesterol; about 9% of purified glycerylcaldityl tetraether; and about 1% to about 15% of a permeability enhancer, selected from the group consisting of cholylsarcosine, octadecanethiol, and D-α-tocopheryl polyethylene glycol 1000 succinate.

In further embodiments, said liposomes have an average diameter of 100 to 200 nm.

In further embodiments, said liposomes have a polydispersity index of 0.05 to 0.20.

In further embodiments, disclosed herein is a method of treatment of a disease comprising the administration of a therapeutically effective amount of a composition as recited in claim 26 to a patient in need thereof.

In further embodiments, disclosed herein is a method of treatment of a disease comprising the administration of a therapeutically effective amount of a composition as recited in claim 33 to a patient in need thereof.

In further embodiments, disclosed herein is a method of enhancing the permeation of an active pharmaceutical ingredient comprising the administration of a composition as recited in claim 26 to a patient.

In further embodiments, disclosed herein is a method of enhancing the permeation of an active pharmaceutical ingredient comprising the administration of a composition as recited in claim 33 to a patient.

In further embodiments, said permeation is increased by greater than 3-fold.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The term “BCS Class III drug” refers to a drug which is characterized under the Biopharmaceutics Classification System guide for predicting the intestinal drug absorption provided by the U.S. Food and Drug Administration as a low permeability, high solubility drug wherein the drug's absorption is limited by the permeation rate but the drug is solvated quickly.

The term “GIT' refers to the gastrointestinal tract.

The term “liposome” refers to artificially prepared vesicles constructed from phospholipid bilayers. Liposomes can be used for delivery of pharmaceutically active compounds due to their unique property of encapsulating a region of aqueous solution inside a hydrophobic membrane; dissolved hydrophilic solutes cannot readily pass through the lipids. Hydrophobic compounds can be dissolved into the membrane, and in this way liposome can carry both hydrophobic and hydrophilic compounds. To deliver the molecules to sites of action, the lipid bilayer can fuse with other bilayers such as the cell membrane, thus delivering the liposome contents. By making liposomes in a solution of pharmaceutically active compounds (which would normally be unable to diffuse through the membrane) they can be delivered past the lipid bilayer. There are three types of liposomes—MLV (multilamellar vesicles) SUV (Small Unilamellar Vesicles) and LUV (Large Unilamellar Vesicles). These are used to deliver different types of drugs. The term “liposomal composition” refers to an emulsion comprising an aqueous solvent in which liposomes are emulsified. Usually, the content of liposomes in such compositions is not exceeding 10 to 25 vol %. The inner space of such liposomes is usually filled with the same liquid solvent in which the liposomes are dissolved.

The term “permeation enhancer”, “permeation enhancement”, or “cell permeation enhancer” refers to a liposome component, which increases the delivery of a pharmaceutically active compound across a layer of cells or to the interior of a target cell. In certain embodiments, cell permeation enhancement can be measured using the Caco-2 assay described herein. In some instances, the terms “permeation” and “permeability” are used synonymously with respect to the above enhancers.

The term “TPGS” refers to D-α-tocopheryl polyethylene glycol succinate.

The term “PEG” refers to polyethylene glycol.

The term “HLB” refers to hydrophilic/lipophilic balance.

The term “CS” refers to cholylsarcosine.

The term “EPC” refers to egg phosphatidylcholine.

The term “CpCl” refers to cetylpyridinium chloride, a cationic surfactant and permeation enhancer.

The term “SA” refers to stearylamine.

The term “OT” refers to octadecanethiol.

The term “FITC-dextran” refers to a complex, branched polysaccharide made of many glucose molecules, composed of chains of varying lengths, which has been coupled with the fluorescent molecule fluorescein isothiocyanate (FITC).

The term “HPLC” refers to high performance liquid chromatography.

The term “TFA” refers to trifluoroacetic acid.

The term “DMEM” refers to Dulbecco's modified Eagle's medium.

The term “FBS” refers to fetal bovine serum.

The term “LDH” refers to lactate dehydrogenase.

The term “KRB” refers to Krebs-Ringers buffer.

The term “C_{cl}” refers to cell capacitance.

The term “TER” refers to transepithelial electrical resistance.

The term “ANOVA test” refers to an analysis of variance test.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

While it may be possible for the pharmaceutical compositions of the subject invention to be administered as the raw liposomal composition, it is also possible to present them as a pharmaceutical formulation. Accordingly, provided herein are pharmaceutical formulations which comprise one or more of certain liposomal composition disclosed herein, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include preferably those suitable for oral administration although the most suitable route may depend upon for example the condition and disorder of the recipient. The formulations may conveniently be presented in unit dosage form and may be prepared by any of the methods well known in the art of pharmacy. Typically, these methods include the step of bringing into association a liposomal composition of the subject invention (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the liposomal compositions disclosed herein suitable for oral administration may be presented as discrete units such as capsules or cachets each containing a predetermined amount of the active ingredient; as a solution or a suspension in an aqueous liquid or a non-aqueous liquid; or as an oil-in-water liquid emulsion or a water-in-oil liquid emulsion. The active ingredient may also be presented as a bolus, electuary or paste.

The liposomal compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and nonaqueous (oily) sterile injection solutions of the active liposomal compositions which may contain antioxidants, buffers, bacteriostats and solutes which render the formulation isotonic with the blood of the intended recipient; and aqueous and nonaqueous sterile suspensions which may include suspending agents and thickening agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the liposomal compositions to allow for the preparation of highly concentrated solutions.

In addition to the formulations described previously, the liposomal compositions may also be formulated as a depot preparation. Such long acting formulations may be administered by implantation (for example subcutaneously or intramuscularly) or by intramuscular injection. Thus, for example, the liposomal compositions may be formulated with suitable polymeric or hydrophobic materials (for example as an emulsion in an acceptable oil) or ion exchange resins, or as sparingly soluble derivatives.

Certain liposomal compositions disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, liposomal compositions may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the liposomal compositions according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Liposomal compositions may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more liposomal compositions which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The liposomal compositions can be administered in various modes, e.g. orally, topically, or by injection. The precise amount of liposomal composition administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the liposomal compositions described herein in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the liposomal compositions described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the liposomal compositions described herein with another therapeutic agent (which also includes a therapeutic regimen) that also has therapeutic benefit. By way of example only, in a treatment for diabetes involving administration of one of the liposomal compositions described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating disorders in a human or animal subject in need of such treatment comprising administering to said subject an amount of a liposomal composition disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment disorders.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

The figures show:

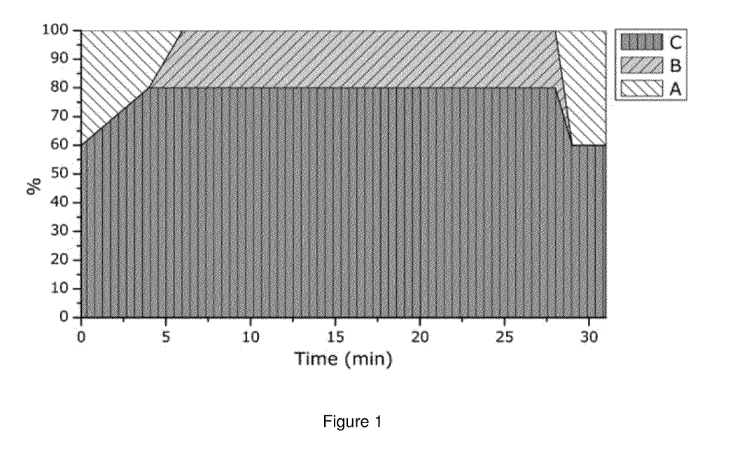

FIG. 1:

HPLC gradient with following mobile phases: water+0.05% TFA (phase A), methanol+0.05% TFA (phase B) and acetonitrile+0.05% TFA (phase C).

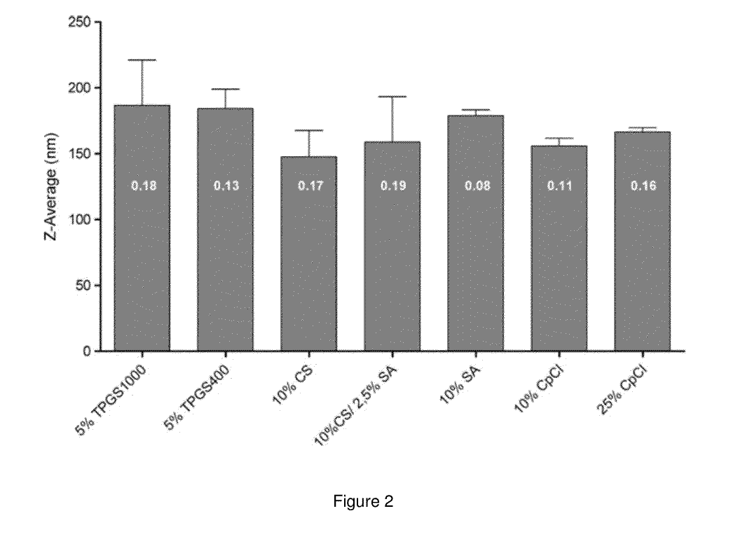

FIG. 2:

Z-Average and polydispersity index (marked in white on the size bars) of the liposomal formulations for the permeation studies. The size is expressed in nm and given as means±SEM with n=3.

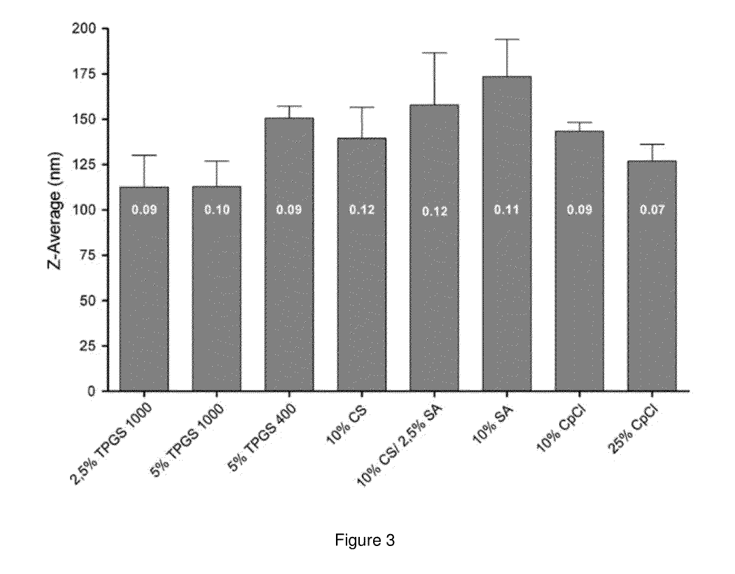

FIG. 3:

Z-Average and polydispersity index (marked in white on the size bars) of the liposomal formulations for the toxicity studies. The size is expressed in nm and given as means±SEM with n=3.

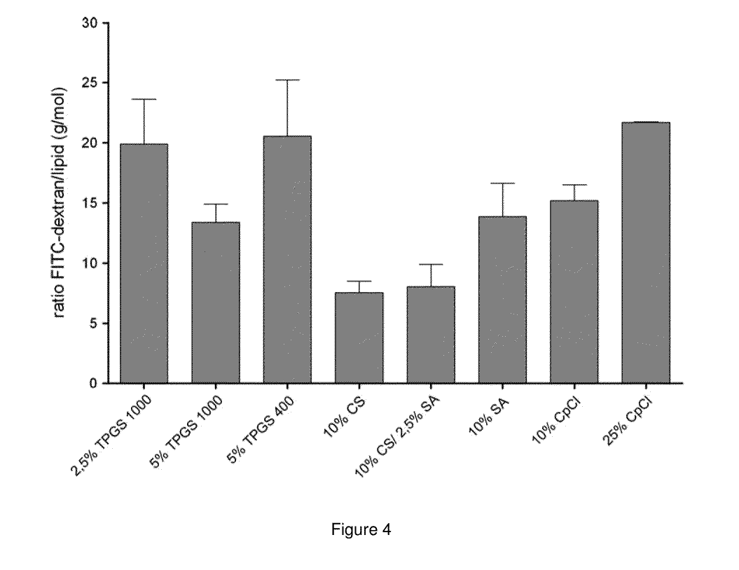

FIG. 4:

Ratio of FITC-dextran to the total lipid amount in the liposomes after purification. The ratio is expressed in mass FITC-dextran/total lipid amount (g/mol) and given as means±SEM with n=3.

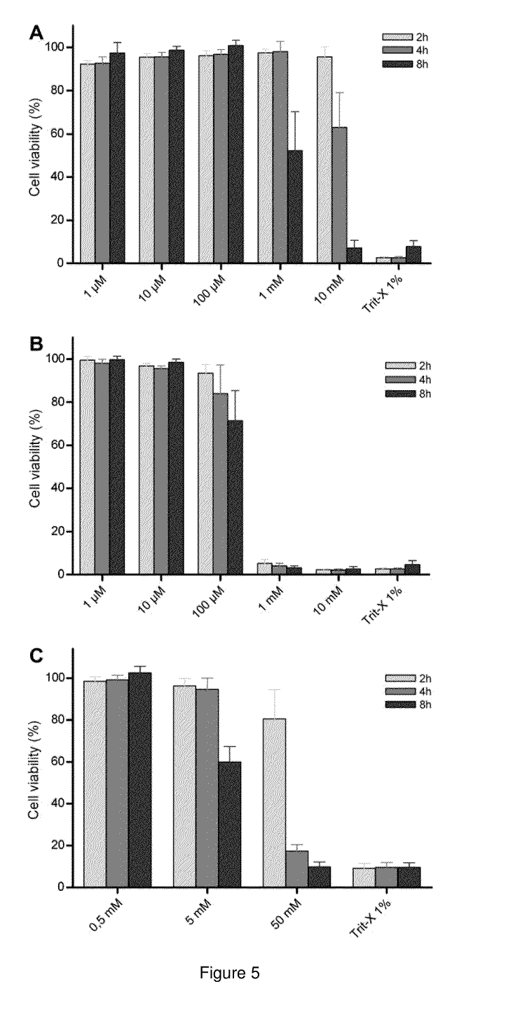

FIG. 5:

Cell viability in the Alamar Blue® assay after 2-, 4- and 8-h incubation with (A) TPGS 1000 in KRB, (B) CpCl in KRB and (C) liposomes with 25% CpCl in KRB. Bars represent cell viability in % and are given as means±SEM with n=3.

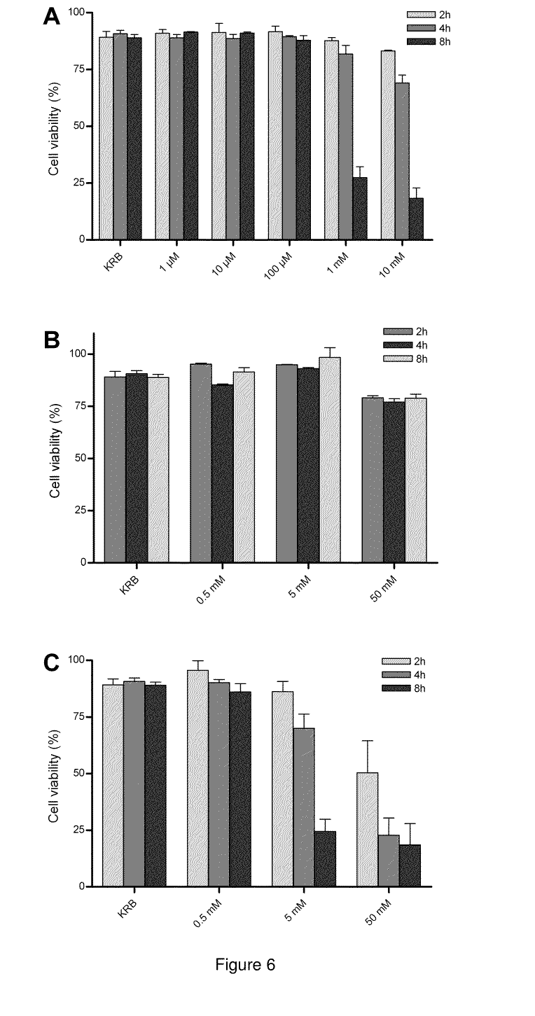

FIG. 6:

Cell viability in the LDH assay after 2-, 4- and 8-h incubation with (A) TPGS 1000 in KRB, (B) liposomes with 10% CS and 2.5% SA in KRB and (C) liposomes with 25% CpCl in KRB. Bars represent cell viability in % and are given as means±SEM with n=3.

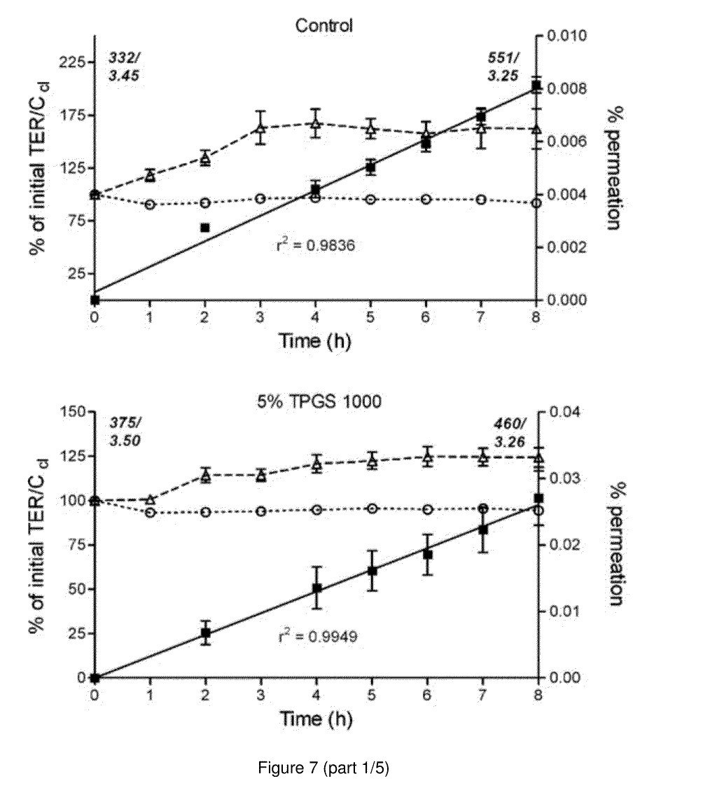

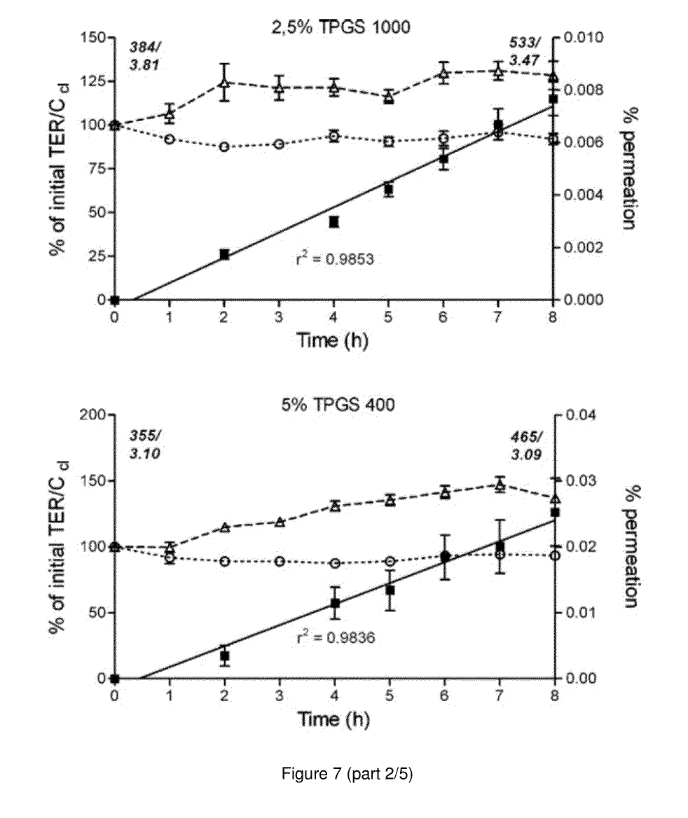

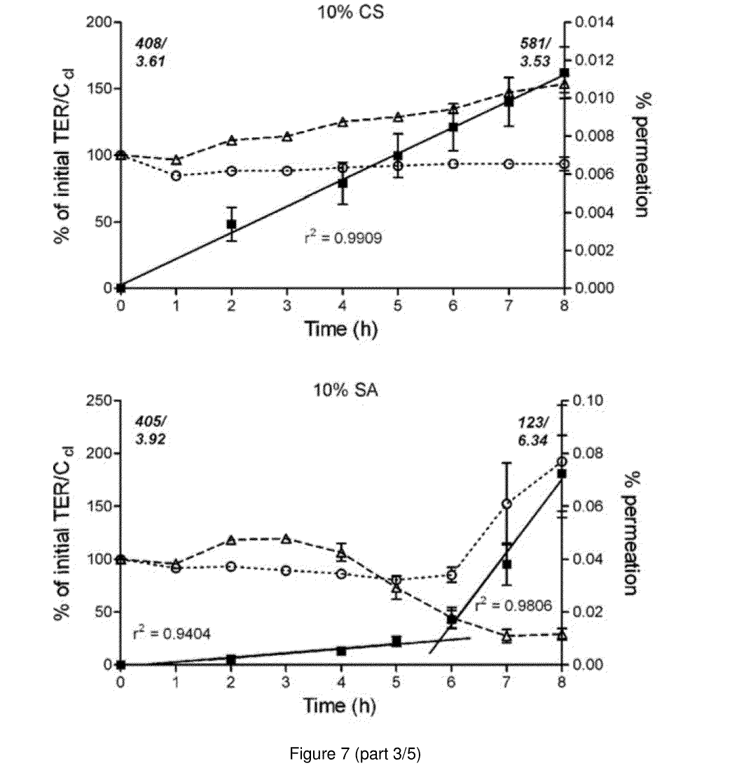

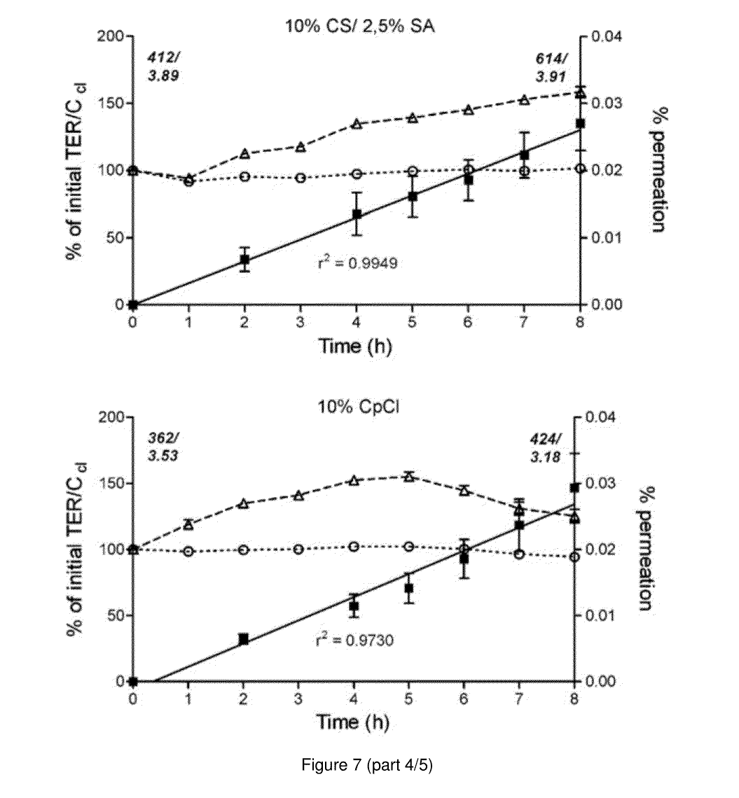

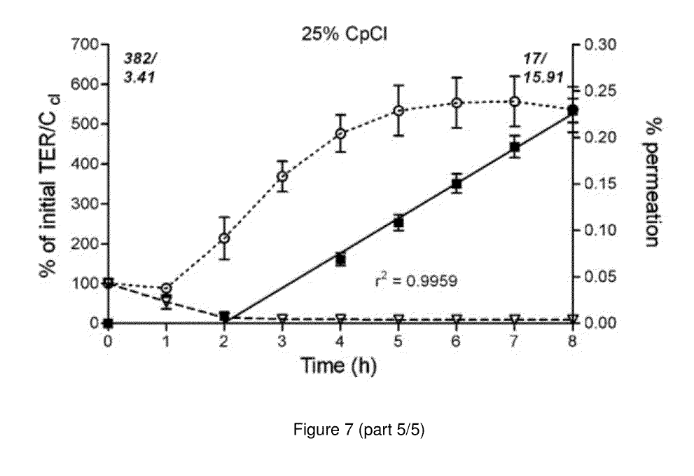

FIG. 7:

The graph shows the development of TER (open triangles, left axis) and C_{Cl} (open circles, left axis) in % of the initial value and the permeation of FITC-dextran (closed squares, right axis) in % of the applied dose on the apical side. The values are given as means±SEM with n=8. Absolute values of TER in Ω/cm² (first value) and C_{Cl} in μF/cm² (second value) at the beginning of the experiment (left side) and the end (right side) are labelled in the graphs.

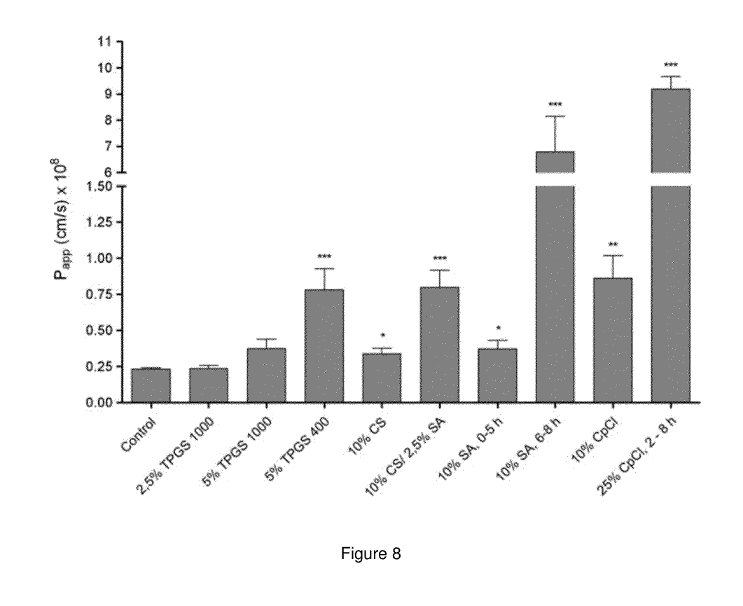

FIG. 8:

Pₐₚₚ of FITC-dextran as free control and encapsulated in liposomes. The Pₐₚₚ is expressed in (cm s⁻¹)×10⁸ and given as means±SEM with n=8. Control and treatment groups were compared by one-way Student's t-test with p*<0.05, p**<0.01, p***<0.001.

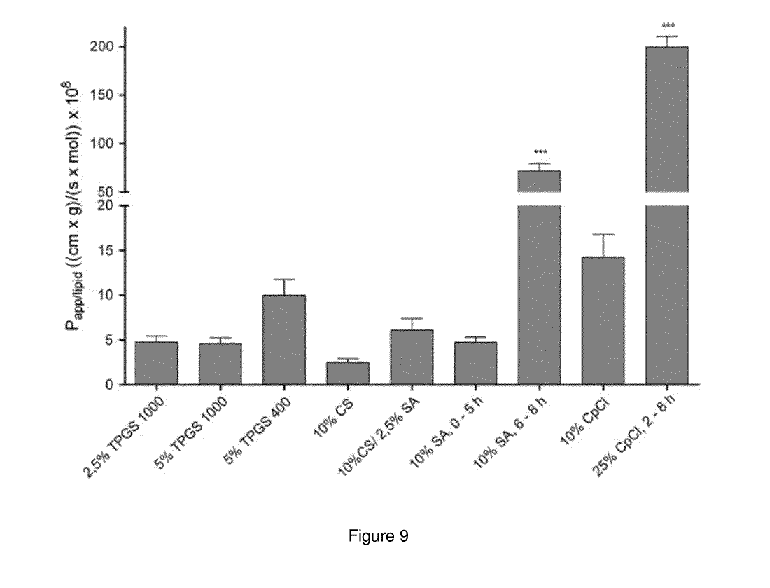

FIG. 9:

Virtual P_{app/lipid} of the total lipid amount. The P_{app/lipid} is expressed in (cm g)×(s mol)⁻1×10⁸ and given as means±SEM with n=8. Values were compared by one-way ANOVA test with p*<0.05, p**<0.01, p***<0.001.

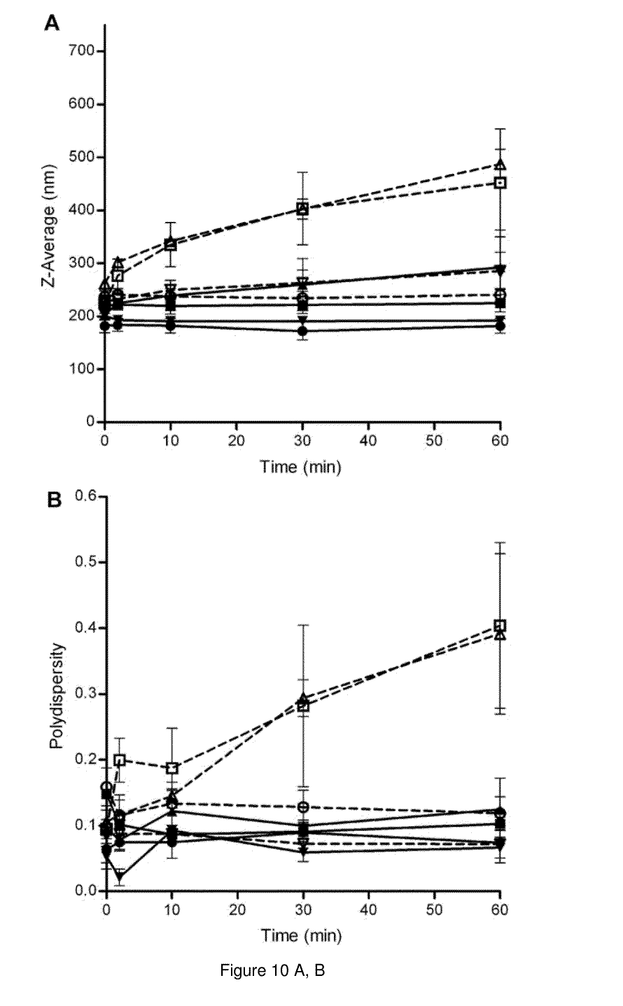

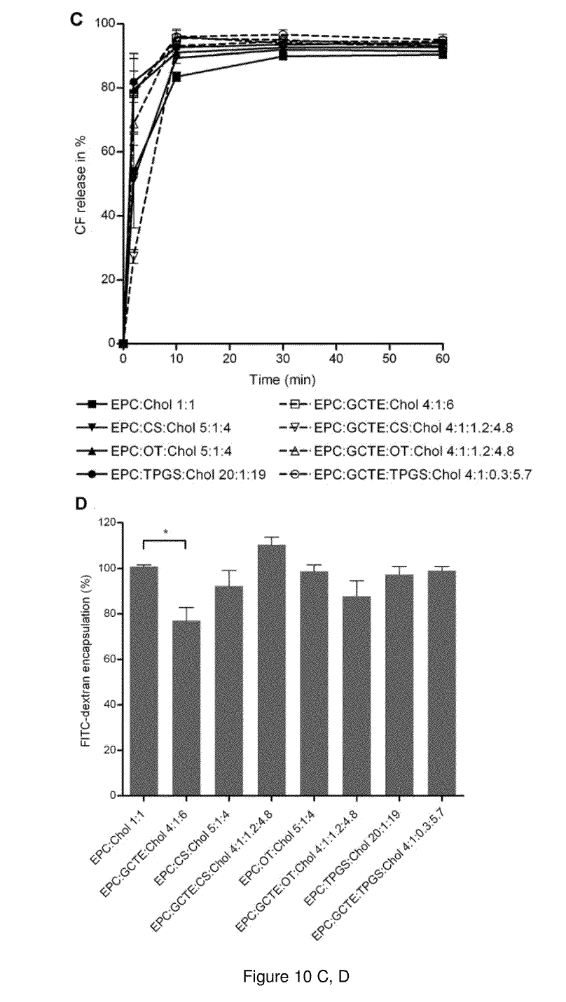

FIG. 10:

Size (A), polydispersity index (B), CF release (C) and FITC-dextran 70 kDa encapsulation (D) after 60 min of liposomes in Tris buffer pH 2 at 37° C. Values represent mean±SEM with n=4 (A and B) or n=3 (C and D). Groups with and without GCTE in graph D were compared by one-way Student's t-test with *p<0.05, p<0.01, p<0.001.

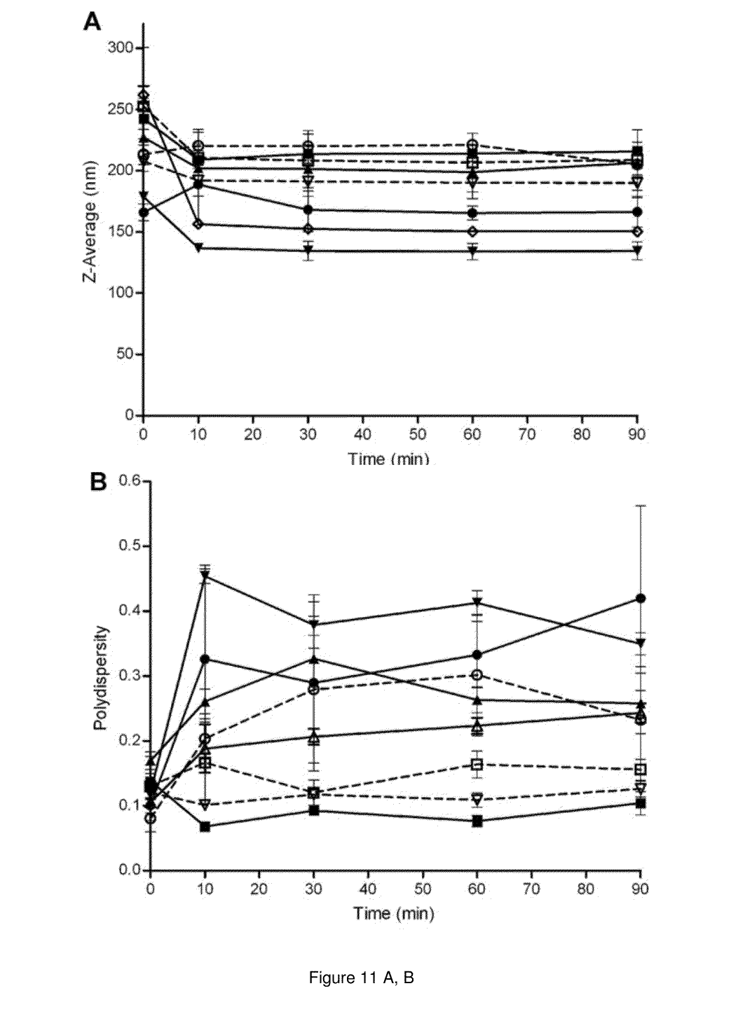

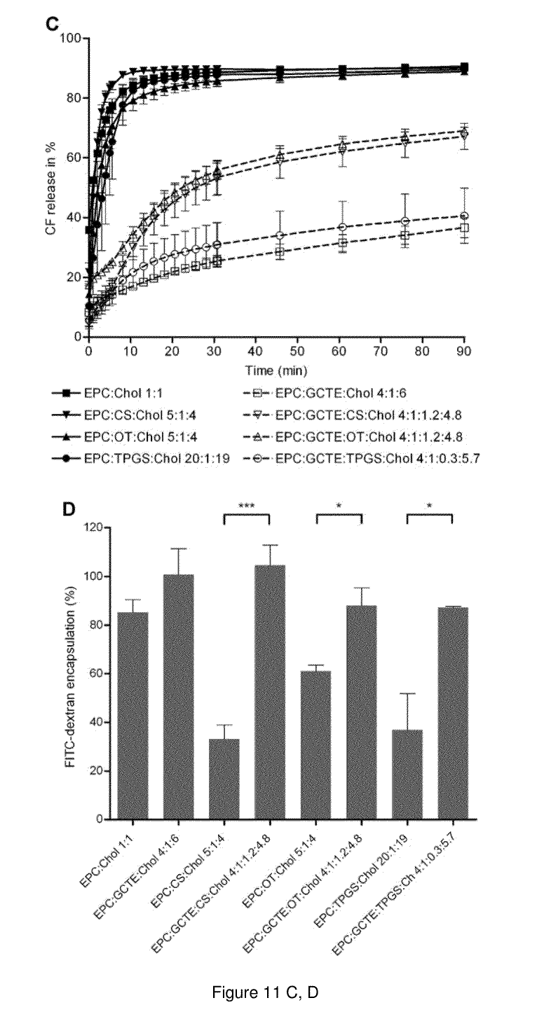

FIG. 11:

Size (A), polydispersity index (B), CF release (C) and FITC-dextran 70 kDa encapsulation (D) after 90 min of liposomes in sodium taurocholate 10 mM at 37° C. Values represent mean±SEM with n=4 (A and B) or n=3 (C and D). Groups with and without GCTE in graph D were compared by one-way Student's t-test with *p<0.05, p<0.01, ***p<0.001.

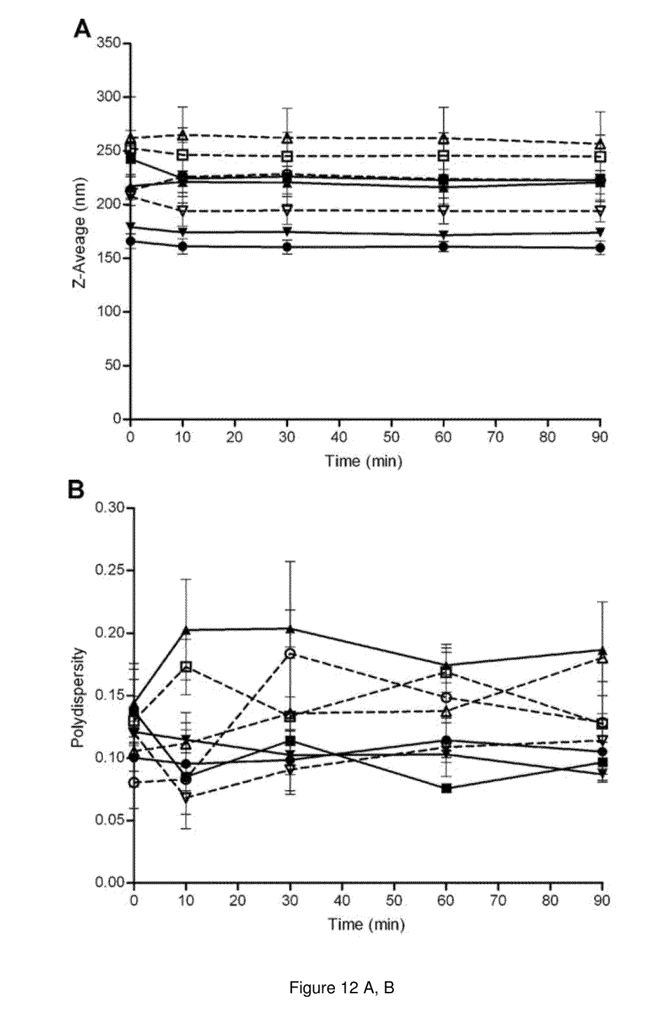

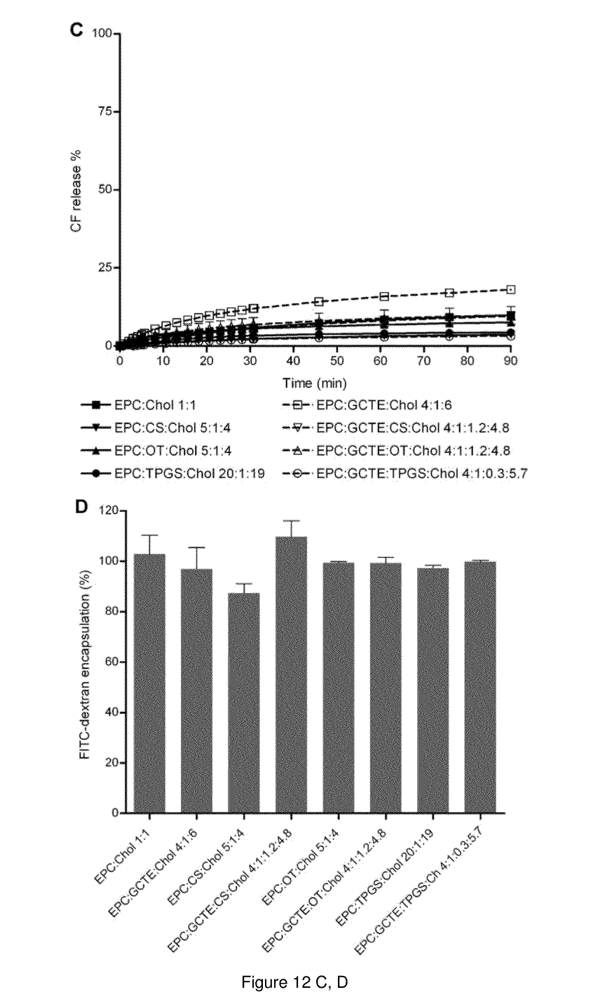

FIG. 12:

Size (A), polydispersity index (B), CF release (C) and FITC-dextran 70 kDa encapsulation (D) after 90 min of liposomes in pancreatin 0.3% 8×USP at 37° C. Values represent mean±SEM with n=4 (A and B) or n=3 (C and D). Groups with and without GCTE in graph D were compared by one-way Student's t-test with p<0.05, p<0.01, p<0.001.

The present invention will be further illustrated in the following examples without any limitation thereto.

### EXAMPLES

### Methods for Preparing Compositions

### Materials

EPC was provided by Lipoid GmbH (Ludwigshafen, Germany). GCTE was provided by Bernina Plus GmbH (Planegg, Germany). TPGS 1000 and TPGS 400 were supplied by Eastman (Kingsport, Tenn., USA). CpCl was purchased from Roth (Karlsruhe, Germany). CS was obtained from Prodotti Chimici e Alimentari S.p.A (Basaluzzo, Italy). Cholesterol, SA, fluorescein isothiocyanatedextran (Mw 70000 Da) (FITC-dextran), pancreatin from porcine pancreas (8×U.S.P.), octadecanethiol and sodium taurocholate (minimum 95% TLC) were purchased from Sigma-Aldrich (Taufkirchen, Germany). Culture media, fetal bovine serum (FBS) and supplements were purchased from Biochrom (Berlin, Germany). 5(6)-Carboxyfluorescein was provided by Serva (Heidelberg, Germany). All other chemicals were obtained in the highest purity from the usual commercial sources.

Pancreatin mixture contains non-soluble components, which could disturb the fluorescence measurements. In order to remove these impurities, 1.25% (m/m) of pancreatin was dispersed in phosphate buffered saline (PBS) (NaCl-137 mM, KCl 2.7 mM, K₂HPO₄.1.5 mM, Na₂HPO₄.2H₂O 8.1 mM) and centrifuged at 15,000×g for 1 h at 4° C. (Beckman J2-MC, Beckman Instruments GmbH, Munich, Germany). Finally, the supernatant was filtrated using a 0.45 μm sterile filter. The lipase activity was determined according to the assay described in Ph. Eur. 6.3. using a Dosimat E 412 (Metrohm Herisan, Metrohm GmbH, Filderstadt, Germany) for titration and a pH-Meter E 512 (Metrohm Herisan, Metrohm GmbH, Filderstadt, Germany) for pH measurement. The pancreatin solution was further diluted with PBS 1:4 to achieve a lipase activity of 300 U/ml and stored at −80° C. until use.

### Preparation of Liposomes

The different enhancers were mixed with EPC and cholesterol, whereby EPC was always 50% (mol/mol) of the lipid mixture and the enhancers and cholesterol summed up to the other half of the formulation, wherein the enhancers were either 10% (CS and OT) or 2.5% (TPGS). The liposomes were prepared by the film method according to Bangham et al. [22]. Therefore, the lipids were dissolved in chloroform/methanol (9:1) and mixed in a 5-ml glass vial in the desired ratio. The solution was dried under a nitrogen stream and kept under a high vacuum for 2 h to remove any solvent traces.

The films were either hydrated with CF 50 mM in PBS or FITC-dextran 20 mg/ml in PBS or with PBS without any marker to achieve a final total lipid concentration of 10 mM (CF and PBS liposomes) or 100 mM (FITC-dextran liposomes). Subsequently the liposomes were extruded 21 times through a 200 nm polycarbonate membrane using a LiposoFast extruder (Avestin, Ludwigshafen, Germany). Size and polydispersity were checked by dynamic light scattering (DLS) using a Zetasizer 3000 HS (Malvern Instruments GmbH, Herrenberg, Germany) in the intensity mode.

In terms of the cytotoxicity studies, the film was hydrated with Krebs-Ringer-Buffer (KRB) (NaCl 142 mM, KCl 3 mM, K2HPO4.3 H2O 1.5 mM, HEPES 10 mM, D-glucose 4 mM, MgCl2. 6 H2O 1.2 mM and CaCl2.2 H2O 1.4 mM) to a final concentration of 150 pmol/ml lipid dispersion and the vesicle were extruded five times through an 800-nm membrane and 15 times through a 200-nm membrane using a Lipex™ extruder (Northern Lipids, Burnaby, BC, Canada).

For the permeation assay, the lipid film was hydrated with FITC-dextran 20 mg/ml in KRB to a lipid concentration of 200 pmol/ml. The dispersion was then sonicated in a bath type sonicator for 2 h (Elmasonic S 300 H, Elma®, Singen, Germany) and extruded 41 times through a 100-nm membrane using a LiposoFast extruder (Avestin, Ludwigshafen, Germany). The liposomes were separated from the non-encapsulated marker over a Sepharose® CL-4B column, and the amount of the encapsulated FITC-dextran was determined by its fluorescence at an excitation wavelength of 485 nm and an emission wavelength of 520 nm in a Fluoroskan Ascent® plate reader (Thermo Fischer Scientific, Waltham, Mass., USA) against a calibration curve. The liposomes were further diluted to a FITC-dextran concentration of 0.5 mg/ml.

The final lipid concentration of the liposomes was determined by HPLC. Size and polydispersity were checked by photon correlation spectroscopy (PCS) using a Zetasizer 3000 HS (Malvern Instruments GmbH, Herrenberg, Germany).

### HPLC-lipid Analysis

The liposomes were diluted 1:10 in methanol, preincubated at 35° C. for at least 30 min and then injected by an autosampler (35° C.) in a Dionex UltiMate® 3000 system (Dionex, ldstein, Germany) with a UV PDA detector and an Acclaim® 120 C18 5 μm column (4.6 mm 250 mm) at 45° C. The mobile phase consisted of the following solvents: water+0.05% trifluoroacetic acid (TFA) (phase A), methanol+0.05% TFA (phase B) and acetonitrile+0.05% TFA (phase C). The flow was kept at 1.2 ml/min throughout the run. The gradient was programmed as shown in FIG. 1. The concentration of the substances was determined by comparing the UV absorption at 215 nm to a calibration curve.

### Biological Activity Assays

### Cell Culture

Caco-2 cells were grown in T-75 flasks in Dulbecco's modified Eagle's medium (DMEM) supplemented with 10% FBS, 1% non-essential amino acids, 1% pyruvate, 1% L-glutamine, 100 U/ml penicillin and 100 μg/ml streptomycin at 37° C. in an atmosphere with 5% CO₂ and in equilibrium with distilled water. The medium was changed every other day, and the cells were subcultured at 80% confluency. Cells were used in the experiments described below.

### Cytotoxicity Assays

Cytotoxicity of liposomes and bio-enhancer solutions was investigated using the Alamar Blue® assay (AbD Serotec, Oxford, UK) and by determination of the release of lactate dehydrogenase (LDH) with a test kit (Sigma-Aldrich, Taufkirchen, Germany). The Alamar Blue® assay is based on the ability of mitochondrial dehydrogenase to cleave the tetrazolium rings of a blue MIT derivative (3-(4,5-dimethylthiazol-2-yl)-2,5-diphenyl-tetrazolium bromide), whereby a pink-coloured formazan product is formed. The LDH assay relies on the reduction of NAD by LDH, which releases from the cells due to cell membrane damage. The formed NADH in turn reduces a tetrazolium salt to a red-coloured product which can be determined photometrically.

Caco-2 cells were seeded onto rat tail collagen (Roche, Mannheim, Germany) coated 96-well plates at a density of 65,000 cells/cm² and grown for 14 days under the conditions described above. Twelve hours prior to experiments, the growth medium was changed to serum, antibiotics and phenol red-free DMEM, and the cells were finally washed twice with KRB.

In terms of the Alamar Blue® assay, groups of eight wells were incubated with the solutions of the bio-enhancers in five concentrations (1 μM, 10 μM, 100 μM, 1 mM and 10 mM) and the liposomal formulations in three concentrations (0.5 mM, 5 mM and 50 mM total lipid) in KRB for 2, 4, and 8 h at 37° C. Due to its poor water solubility, SA was dispersed in KRB using a tip sonicator (Soni 130, G. Heinemann, Schwabisch Gmünd, Germany) for 5 min at 130 W and only tested in three concentrations (1 μM, 10 μM and 100 μM). After incubation, the cells were washed twice with KRB and an Alamar Blue® solution in KRB (1:80) was added. After 4 h at 37° C., the colour change of the dye was determined at an excitation wavelength of 530 nm and an emission wavelength of 590 nm in a Fluoroskan Ascent® plate reader. Cells incubated with KRB were used as untreated control, and cells exposed to 1% Triton X-100 were used as positive control. Cell viability was expressed as follows:

% Cell viability=Sample value/Untreated control×100% (1)

For the LDH release assay, cells were treated similar as described for the Alamar Blue® cytotoxicity assay with little changes. A 25-μl sample was withdrawn from the cell supernatant after 2, 4, and 8 h, diluted with the same volume of KRB and was incubated with 50 μl of the assay mixture for 30 min in the dark as recommended by the manufacturer. The reaction was stopped by the addition of 10 μl 1N HCl and subsequently, the colour change of the dye was measured by a Tecan microplate absorbance reader (Sunrise, Tecan, Grodig, Austria) at a wavelength of 490 nm and a reference wavelength of 700 nm. To eliminate the influence of the liposomes and the bio-enhancers on the absorbance, a blank was measured of the formulations with a concentration equal to the final assay concentration. Cell viability was calculated by the following equation:

% Cell Viability=(A_{Triton 1%}−(Aₛₐₘₚₗₑ−A_{blank}))/A_{Triton 1%}×100% (2)

where A_{Triton 1%} is the absorbance after incubation with 1% Triton X-100, Aₛₐₘₚₗₑ the absorbance after incubation with the test solutions, and A_{blank} the absorbance of the test solutions without the addition of the assay dye mixture.

### Permeation Studies

Caco-2 cells were seeded at a density of 75,000 cells/cm² onto rat tail collagen-coated 12 Transwell® polyester filters with 0.4-μm pore size (Corning, Kaiserslautern, Germany) and grown for 21 days under the conditions described above. Twelve hours prior to experiments, the growth medium was changed to serum, antibiotics and phenol red-free DMEM and the cells were finally washed twice with KRB. The transport experiments were performed in a device (cellZscope®, Nanoanalytics, Munster, Germany) monitoring automatically the transepithelial electrical resistance (TER) and the cell capacitance (C_{cl}) of 24 filters in parallel by measuring the frequency-dependent impedance of the cell layer. The theoretical background of the impedance measurement is described in detail by Wegener et al. [23]. The filter inserts were placed inside the cellZscope®, and 0.6 ml KRB in the apical (A) and 1.0 ml KRB in the basolateral (B) compartment were added. The cells were allowed to equilibrate for 2 h before the first measurement of TER and C_{cl}, which were monitored throughout the entire experiment every hour. Directly after the first measurement, the KRB on the A-side was replaced with the liposomal dispersion or the solution of the free FITC-dextran. Samples of 200 μl were withdrawn every hour from the B-side, replaced with KRB and transferred in a black 96-well plate (Corning, Kaiserslautern, Germany). The concentration of the FITC-dextran was determined as described above. The apparent permeation coefficient (Pₐₚₚ) of dextran was calculated following the equation:

Pₐₚₚ=dQ/dt·1/(A·C₀)·cm/s (3)

where C₀ is the concentration of FITC-dextran on the A-side (μg/cm³) at time point zero, and A is the total surface area of the filter (cm²). dQ/dt was calculated by the slope of the linear range of the permeation rate of FITC-dextran (μg/s).

### Performance of Liposomes Depending on Encapsulation Efficiency

In order to compare the performance of the different liposomes depending on their encapsulation efficiency, a virtual permeation coefficient for the liposomes was calculated by relating the apparent permeation coefficient of the FITC-dextran to the encapsulation efficiency of the liposomes. The apparent permeation coefficient (P_{app/lipid}) was calculated as follows:

P_{app/lipid}=Pₐₚₚ·C₀/C_{1ipid}·(cm g)/(s mol) (4)

where C₀ is the concentration of the dextran on the A-side (μg/cm³), and C_{lipid} is the total lipid concentration on the A-side (μmol/cm³).

### Statistics

All values are presented as means±SEM. Control and treatment groups were compared by one-way Student's t-test or one-way ANOVA test as indicated in the figures. Differences were considered significant at p*<0.05, p**<0.01, p***<0.001. Plots and statistical analysis were made using the software Prism® (GraphPad Software, San Diego, Calif., USA).

### Liposome Properties

Size and polydispersity of the liposomes are shown in FIGS. 2 and 3. All formulations were in size between 110 nm and 190 nm and showed a narrow distribution indicating the successful formation of liposomes. Although the vesicles for the toxicity studies were extruded through a 200-nm membrane, they were mostly similar in size to the liposomes for the transport studies, probably due to the higher flow rate of the Lipex™extruder [24].

FIG. 4 shows the ratio of the marker FITC-dextran to the total lipid amount in the liposomes after the purification step. To achieve a final marker concentration of 0.5 mg/ml, the liposomes were finally diluted to a total lipid concentration between 15 and 40 mM depending on the encapsulation efficiency of the liposomal formulation.

Whereas SA is used rather often in liposomes to obtain a positive surface charge, there is little use of CpCl and to our knowledge, no use of TPGS and CS in liposomes [10, 25-28]. However, there are at least two studies using other bile salts in liposomes for permeation enhancement [16, 17]. The cautious use of surfactants in lipid vesicles might be related to their potentially destabilizing effect on liposomes. Besides the packing constraints of the lipid bilayer, steric conditions and the curvature of the vesicles, this is mostly dependent on the critical micelle concentration (CMC) of the surfactant, whereas electrostatic interactions have an inferior influence [29]. The CMC values for the different enhancers at room temperature found in the literature are as follows: CpCl 0.98 mM, CS between 10 and 11 mM, TPGS 1000 around 0.02 mM and TPGS 400 around 1.5 mM [13, 30, 31]. For SA, no CMC could be found in the literature. Usually, when the CMC of a surfactant increases, also the concentration needed to form mixed micelles with phospholipids is increased [29]. Surprisingly, TPGS 400 has a higher CMC compared to TPGS 1000 even though it has a lower HLB (8.3 vs. 13.2) [32]. However, the smaller size and the lower encapsulation efficiency of the formulation with 5% TPGS 1000 indicate that indeed TPGS 1000 in the chosen concentration forms not just mixed vesicles but also mixed micelles. This does not apply to TPGS 400 or at least to a lower extent apparent by the bigger size of the liposomes formed by the LiposoFast extruder. The CS-containing vesicles for the cytotoxicity studies were remarkably smaller than the others. Despite the high CMC of CS, the smaller size and the very poor encapsulation efficiency of both of the CS containing formulations imply again the formation of mixed micelles. It is unlikely that the small FITC-dextran/lipid ratio is only caused by the negative charge of CS, since there is no difference in the ratio for the formulation containing both the positively charged SA and CS. In terms of the manually extruded vesicles, the liposomes with 10% SA were slightly bigger compared to the other formulations. This effect was also found by Zschriinig et al. [33]. The liposomes with 25% CpCl showed the highest encapsulation efficiency though they were comparable in size to the other formulations. This may indicate that the cationic CpCl interacts with the carboxylic groups of the fluoresceine isothiocyanate and binds to some extent the fluorescent marker to the lipid bilayer.

Summarising the results of the liposome characterisation, the data show that it is possible to form liposomes containing surfactants in different ratios. However, the influence of temperature changes, dilution of the formulations and other in vivo conditions were not examined in this study.

### Cytotoxicity Studies

Most of the tested enhancers and all of the liposomal formulations with one exception showed no toxicity in the tested concentrations in both assays. Altogether, the assays led to similar results; whereas the LDH release after incubation with the CpCl solution in all concentrations seemed to be lower as the release after incubation with KRB, it is likely that the free CpCl can inhibit the enzymatic reaction and finally the formation of the red tetrazolium dye. On the other hand, this effect could not be observed for the two liposomal formulations containing CpCl, probably because the amount of free CpCl is very low in case of the liposomal dispersions.

Cell viability for the formulations, which showed a toxic effect, is displayed in FIGS. 5 and 6. In the Alamar Blue® assay, CpCl exhibited at a concentration of 1 mM already a strong cytotoxicity and TPGS 1000 reduced the cell viability at a concentration of 10 mM after 8-h incubation to a minimum, whereas no effect could be observed after 2-h incubation time. This was also confirmed by the LDH release. The liposomes with 25% CpCl had a strong toxicity in a total lipid concentration of 50 mM after 4-h incubation. However, by reducing the CpCl amount to 10%, any toxic effect in the tested concentration could be avoided, although the total CpCl amount of 50 mM liposomes with 10% CpCl is higher than for the 5 mM liposomes with 25% CpCl, indicating that the toxic effect is not just correlated with the total amount of the surfactant but also with its concentration in the liposomal membrane. The SA/CS liposomes led in all tested concentrations after 8 h to a slightly higher LDH release compared to the KRB control. This effect was not visible in the Alamar Blue® assay.

The cytotoxicity of surfactants is dependent on their charge, chemical characteristics and their CMC, but also on the cells and the assay used for determining the toxicity [34-36]. Often cationic compounds show a higher toxicity compared to uncharged or negatively charged molecules. As a matter of fact, CpCl was found to be the most toxic of the investigated enhancers. Burgalassi et al. found in their study with two different corneal cell lines a decrease in the cell viability of 50% after 1-h incubation with CpCl in a concentration of around 10 μM [37]. In a fibroblast cell line, the concentration of CpCl leading to 50% survival after 30 min was determined at around 0.19 mM with two different assays [38]. In a further study, the cytotoxicity of CpCl was comparable to that of the anionic surfactant sodium dodecyl sulphate concerning its haemolytical activity in erythrocytes and protein leaching of nasal mucosa [18]. The somehow lower toxicity found in the present study might be elated to a lower susceptibility of Caco-2 cells to the cationic surfactant. The LD₅₀ was determined at 200 mg/kg in rats and at 400 mg/kg in rabbits [39]. Moreover, there are mouthwashes with CpCl in the market and throat lozenges with a daily maximum intake of 10 mg (Dobendan Strepsils®), which are approved in Germany. Furthermore, by comparing the toxicity of the 10% CpCl liposomes with the free substance, a reduction in toxicity of around 50-fold can be observed.

In an animal study, rats were fed daily amounts of around 37.7 mg/kg SA for two years without causing any side effects [40]. Several studies investigating the cell toxicity of liposomes containing SA can be found [27, 41, 42]. In one study, a growth inhibition of 50% was shown by 10% SA liposomes in Caco-2 cells already at a total lipid concentration of 0.05 mM. However, in this study, the cells were incubated already one day after seeding for 6 days. In this early state, the cells are more susceptible to growth inhibition because they are not organised in a tight cell layer. Furthermore, the long incubation time differs from our methods explaining the different findings.

The higher toxicity of TPGS 1000 compared to TPGS 400 might be again related to the lower CMC of TPGS 1000. As described before, a lower CMC implies a higher cytotoxicity. Collnot et al. found in their study in Caco-2 cells in a LDH release assay for TPGS 400 no toxic effect up to a concentration of 10 mM and for the TPGS 1000, a toxicity starting at a concentration of 625 μm, which is in good conformity with our results [43]. In the literature, a LD50 of >7000 mg/kg rat is described, and a daily intake of 1000 mg/kg of TPGS 1000 and more than 1000 mg/kg of TPGS 400 is considered as safe, suggesting a very good safety profile [11, 44].

In a previous study, we found no cytotoxicity for CS in a WST-1 transformation assay and just a slight LDH release in a concentration of 10 mM in Caco-2 cells confirming the results found in the present study [45]. Several in vivo studies in rodents and also humans suggest a good safety of CS [13, 46-48]. Especially, the low N-demethylation in the sarcosine group going along with a marginal dehydroxylation indicates a low carcinogenic potential.

To a certain extent, a relation between the efficacy of an enhancer and its toxicity can be assumed and is also described in the literature [36, 49]. However, for daily therapeutical use, toxic side effects have to be avoided without diminishing the desired enhancing effect. Depending on overall toxicity and mode of action of the enhancer type, the safe but effective concentration range can be reached more or less reliable. Especially, the two TPGS derivatives used in the present study are orally well tolerated, and the maximum daily intake suggested by the authorities is far above the amount necessary for the use as excipient and more related to their originally intended application for treatment of vitamin E deficiency. On the other hand, the use of the more toxic cationic enhancers in oral delivery systems has to be carefully considered until more well-founded data concerning a safe maximal daily intake are available. Still, the advantage of liposomal systems containing enhancing substances is the ability to reduce the amount of enhancer necessary to achieve the desired effect, as they are co-delivered with the drug to the mucosa.

### Permeation Studies

The permeation rate of FITC-dextran and the development of the TER and the C_{Cl} for the different formulations are shown in FIG. 7. For most of the formulations, an increase in the TER over the time could be observed, whereas the capacitance was stable during the experiment. The vesicles with 25% CpCl reduced the resistance nearly down to zero after 3 h and increased the CCl around fivefold. Considering the cell toxicity of the CpCl liposomes found in the toxicity assays, the change in the TER is probably the result of a toxic effect of the vesicles due to an interaction of the cationic surfactant with the cell membrane causing a disintegration of the membrane structure and a detachment of the cells, visible in the strong increase in the capacitance. This was also confirmed by microscopical examination of the filter inserts after the experiment. The formulation with 10% SA, which showed no toxicity, had a similar influence on the TER and the C_{Cl} but to a much lower extent. Since the surface area of the apical membrane is much greater than that of the basolateral membrane, the capacitance of the total cell layer is mostly dominated by the latter. The doubling of the capacitance can be explained by the opening of the tight junctions and an enlargement of the membrane at the basolateral side [50].

The permeation rate of FITC-dextran was linear for all the formulations but for the two influencing the TER. For the 10% SA, two linear ranges, one from 0 h to 5 h and one from 6 h to 8 h, could be observed. The permeation for the liposomes with 25% CpCl was linear beginning after 2 h.

The apparent permeation coefficient of FITC-dextran as free control and encapsulated in the different liposomal formulations is shown in FIG. 8. Corresponding to the drop in the TER, the 25% CpCl could increase the permeation by 39.28±2.10-fold. For the time between 6 and 8 h, the vesicles with 10% SA led to a similar enhancement, whereas the formulation had nearly no effect in the first 5 h, indicating a strong correlation between the resistance of the cell layer and the permeation of the marker [51, 52]. Unfortunately, this good enhancement went along with a strong toxicity, indicated by the change in TER and C_{Cl}, making those formulations less suitable as drug delivery systems. However, the liposomes with 5% TPGS 400, with the mixture of CS and SA and the formulation with just 10% of CpCl could improve the permeation by 3.34±0.62-fold, respectively by 3.41±0.51 and by 3.69±0.67-fold, with only small or no influence on TER or C_{Cl}. Balda et al. described that it is possible to influence the paracellular permeation of an aqueous marker without changing the electrical resistance of the cell layer [53]. A second explanation could be an endocytosis of the liposomes themselves. As Caco-2 cells lack caveolae, an uptake over clathrin-coated pits or clathrin- and caveolin-independent pathways is conceivable [54, 55]. Theoretically, a fusion of the liposomes with the cell membrane could also contribute to the uptake and permeation of the macromolecule [56]. Furthermore, the direct uptake of the FITC-dextran into the cells could contribute to the permeation through the cell layer, but this event would be far more likely for the free marker compared to the liposomal encapsulated [4, 5, 7].

The bio-enhancing properties of TPGS are mostly referred to the inhibition of P-glycoprotein (P-gp) and the ability to act as a solubiliser of poorly water-soluble drugs [32, 58, 59]. However, the P-gp inhibition as the mechanism of enhancement is controversially discussed and might be just one among several modes of action [59]. Furthermore, FITC-dextran is known to be transported only passively, and the inhibition of P-gp should not influence the permeation of the dextran [20, 21]. It is also discussed whether TPGS 1000 rigidises or fluidises cell membranes [59, 60]. Swenson et al. mentioned that the apical membrane of enterocytes especially in the microvilli is rich in glycolipids and cholesterol leading to a high transition temperature of the lipid bilayer slightly over the physiological body temperature [34]. A change of a gel-state bilayer towards a liquid-crystalline-state bilayer or the other way around always leads to membrane defects during the transition. As no membrane transporters are involved in the permeation of dextran, the TPGS 400 liposomes probably act over an interference with the lipid bilayer of the cells leading to a facilitated uptake of the FITC-dextran or a higher fusion affinity of the liposomes with the cell membrane. The lack of efficacy of the TPGS 1000 in both concentrations might be related to the chain length of the PEG and a possible steric hinderance of the liposome-cell interaction.

In several reviews, the mechanism of the absorption enhancement by bile salts is described as the chelation of calcium ions in lower concentrations and the solubilisation of membrane lipids at higher bile salt concentrations, thus influencing both the paracellular and the transcellular route [34, 35, 61]. Interestingly, CS alone in liposomes could not change the permeation of FITC-dextran, whereas in combination with the cationic lipid SA it could. The ζ-potential of the 10% CS liposomes was negative (−3.93 mV±0.37), but already 2.5% SA changed the potential to a slightly positive value (3.42 mV±1.58). It seems likely that a negative surface charge of the liposomes makes a direct membrane interaction more difficult and that the enhancing effect of the formulation with CS and SA is not due to the SA itself but due to the positive surface charge of the vesicles allowing the CS to interact more efficiently with the Caco-2 cells. SA used alone could only enhance the permeation, when the TER was reduced, which could not be observed with this formulation. As mentioned above, an increase in the paracellular transport by an opening of tight junctions is not always correlated with a change in TER. This means that both pathways of permeation enhancement are theoretically possible for this formulation.

Also the liposomes with 10% CpCl did not change the TER significantly but could increase the permeation of the marker. CpCl showed in previous studies good enhancing effects on both small and large molecules, but the detailed mechanism of enhancement is not clarified yet [18, 62]. Due to the positive charge of the liposomes, an interaction with cell membranes is facilitated. Again, a change in the properties of the membrane bilayer of the Caco-2 cells as mode of action seems likely.

To include the encapsulation efficiency of the vesicles into the analysis of their performance, a virtual permeation coefficient for the lipids was calculated (FIG. 9). Whereas the liposomes containing the mixture of SA and CS were superior to the formulations with just CS and TPGS 1000 concerning the Pₐₚₚ of FITC-dextran, their advantage was diminished regarding the P_{app/lipid} due to their poor encapsulation efficiency. On the other hand, the liposomes with CpCl, which could encapsulate the FITC-dextran very efficiently, needed less lipid to deliver the same amount of marker. However, this effect is linked to the used marker and could be different for other encapsulated substances making a prediction of a possible superiority of the vesicles with CpCl in an industrial scale production difficult.

### Stability Assays

### Dynamic Light Scattering Stability Assay

Liposomes were diluted 1:10 with either Tris buffer pH 2 (Tris 50 mM, KCl 2.7 mM and NaCl 120 mM) or pancreatin in PBS or sodium taurocholate 11.11 mM in PBS—resulting in a final concentration of 10.00 mM sodium taurocholate—and incubated at 37° C. for 60 min, respectively 90 min. A 1:10 dilution of the liposomes in PBS was used as a control. After 10, 30, 60 and in case of the pancreatin and sodium taurocholate assay also after 90 min, a sample was withdrawn, 1:20 diluted and immediately two runs in the rapid mode were performed in the Zetasizer to determine size and polydispersity index (PI).

### Carboxyfluorescein Release

The non-encapsulated CF was separated from the liposomes by a Sephadex® G50 fine size exclusion chromatography. Release of the marker was determined at 37° C. using a Fluoroskan Ascent® (Thermo Fischer Scientific, Waltham, USA) after injection of the liposomes in Tris buffer pH 2, pancreatin in PBS or sodium taurocholate 11.11 mM in PBS resulting in a 1:10 dilution of the formulations. Increase of fluorescence was measured at 485 nm excitation and 520 nm emission wavelength. Because the fluorescence of CF is pH-dependent, the samples were neutralised after 2, 10, 30 and 60 min with Tris buffer pH 10 to achieve a final pH of 7.4. The emission of the liposomes in the mixture of the two different Tris buffers was set as zero release control and the fluorescence in Triton-X 1% in the Tris buffer mix as 100% release control. The emission of CF in the other two assays could be measured continuously and the emission in Triton-X 1% in pancreatin solution, respectively. Triton-X 1% in PBS was set as 100% release. The pancreatin solution has a certain quenching effect on the fluorescence, thus the emission in PBS was only used as a negative control for the test in sodium taurocholate. As the leakage of CF caused by pancreatin is an enzymatic reaction, a 0% release of the marker immediately after liposome injection can be hypothesized. All tests were performed in triplicate in Costar® 24 well plates (Corning, Kaiserslautern, Germany). In these type of wells the influence of the surface tension reduction on the fluorescence by the bile salt and Triton-X is less pronounced than in 96 well plates. The leakage of CF over the time was calculated as follows:

% CF release=(FE−FE₀)/(FE_{Trit}−FE₀)×100% (5)

where FE is the fluorescence emission at the different time points, FE° is the emission of negative control and FE_{Trit} the emission of liposomes after destruction with Triton-X 1%.

### FITC-dextran Release

Liposomes were diluted around 1:4 with PBS and centrifuged at 150,000×g for 90 min at 4° C. (Himac CS 100FX, Hitachi Koki, Tokyo, Japan). Supernatant was removed and liposomes were redispersed in the initial volume of PBS and the centrifugation step was repeated. Directly before the assay, the pellet was dispersed in PBS to achieve a lipid concentration of approximately 50 mM. The formulations were incubated in Tris buffer pH 2, pancreatin in PBS and sodium taurocholate 11.11 mM in PBS at 37° C. for 60 min and 90 min, respectively. Immediately after the incubation the samples were applied on a Sepharose® CL-4B column, eluted with PBS and liposomes and free FITC-dextran were collected separately. The free marker and the liposomes were diluted 1:10 with Triton-X 1% in PBS. Untreated liposomes served as a control. After 1:10 dilution in PBS, they were also separated from any non-encapsulated FITC-dextran by size exclusion chromatography. To determine the recovery rate of the fluorescent marker after the chromatography, non-columned liposomes were diluted 1:10 with Triton-X 1% after 1:10 dilution in PBS. All samples were measured in triplicates in a black Costar® 96 well plate (Corning, Kaiserslautern, Germany). The percentage of encapsulation (E %) was determined by the following equation:

E%=FEₗᵢₚ/(FEₗᵢₚ+FE_{free})×100% (6)

where FEₗᵢₚ is the fluorescence emission of the liposome fraction and FE_{free} of the free marker fraction after correction of the dilution. The percentage of FITC-dextran remaining in the liposomes after incubation in the different buffers in comparison to untreated liposomes was calculated by the following equation:

% FITC−dextran_{remaining}=E%_{treated}/E%_{control×}100% (7)

where E%_{treated} is the percentage of encapsulation in the treated liposomes and E %_{control} in the non-treated liposomes. The recovery rate (RR) in % was calculated as follows:

%RR=(FEₗᵢₚ+FEₜᵣₑₑ)/FE_{uncol}×100% (8)

FE_{uncol} is the fluorescence emission of the uncolumned liposomes. Only samples with a recovery rate between 90% and 110% were taken into account for statistics.

### Statistics

All values are presented as means±SEM. Groups were compared by one-way Student's t-test. Differences were considered significant at *p<0.05, **p<0.01, ***p<0.001. Plots and statistics were made using the software Prism® (GraphPad Software, San Diego, Calif., USA).

### Liposome Stability at pH 2

The majority of the formulations were stable in size and polydispersity over 90 min in Tris buffer pH 2 (FIGS. 10A and B). However, the formulations with EPC/GCTE/Chol and with EPC/GCTE/OT/Chol showed a strong growth in size over the time going together with an increase of Pl. All formulations showed a nearly 100% release of CF after just 10 min at pH 2 (FIG. 10C). In contrast, the encapsulation efficiency of FITC-dextran remained at nearly 100% after 60 min compared to the control for most formulations (FIG. 10D). Only the EPC/GCTE/Chol liposomes were slightly less stable than the same formulation without GCTE.

### Liposome Stability in Sodium Taurocholate 10 mM

After a certain drop in the first minutes, the liposomes were stable in size in the bile salt solutions (FIG. 11A). Also the PI showed the biggest change in the first 10 min, whereas the two formulations without bio-enhancers and the vesicles with GCTE and CS were stable in their size distribution over the time (FIG. 11B). The least stable formulation concerning the PI were the EPC/CS/Chol liposomes with an increase of 3.75-fold. The CF release in bile salts allows a better discrimination of the vesicle stability (FIG. 11C). All liposomes without GCTE released nearly 100% CF in the first 10 min. GCTE vesicles with CS and OT released 67.2% (±4.4) and 69.0% (±1.3), respectively after 90 min, liposomes with GCTE and TPGS 40.6% (±9.3) and GCTE vesicles without enhancer 36.6% (±3.3). FITC-dextran encapsulation exhibited the same inclination of stability, though the release was always lower compared to CF (FIG. 11D). GCTE vesicles retained nearly all of the markers and also EPC/Chol liposomes were remarkably stable containing still 85.3% (±5.2) of FITC-dextran after 90 min. GCTE formulations with bio-enhancers were all significantly more stable than the corresponding formulations without the tetraether lipid.

### Liposome Stability in Pancreatin 0.3% 8×USP

Influence of pancreatin on the stability of the liposomes was very low in all three assays (FIG. 12A-D). Only the CF release varies between the formulations. Most vesicles released less than 10% of the fluorescent dye, only the EPC/GCTE/Chol liposomes were less stable by releasing 18.1% (±0.2) in 90 min, whereas TPGS seemed to have a certain stabilising effect on the CF release.

In the present application, we compared the effects of the tetraether lipid glycerylcaldityl tetraether (GCTE) and various bio-enhancers on the stability of EPC and cholesterol based liposomes. We used a purified, single type TEL, GCTE, to avoid the disadvantages a mixture of TELs entails. It is likely, that the ratio of the different lipid components of archaea membranes varies over the time, making a comprehensive analysis vital. Furthermore, the properties of lipid vesicles can be more easily adjusted and the amount of the TEL needed can be reduced by use of a defined and purified TEL. EPC liposomes with 50% of cholesterol were chosen as reference formulation since the stability of EPC vesicles increases with higher amount of cholesterol, whereas the transition temperature remains below body temperature [100]. Cholesterol formed crystalline structures in the membrane of EPC liposomes at a concentration of around 40%, visible in differential scanning calorimetry scans (data not shown). Thus, a concentration far above 40% is not expected to increase the stability of the vesicles any further, but will complicate handling of the lipid mixture.

Release of the two hydrophilic markers at pH 2 differed very significantly with their molecular weight, indicating that the vesicles were not totally destroyed but a leakage of the small molecule CF through the membrane could occur (see FIG. 100 and D). Also the DLS data indicate that the vesicles stayed intact over the time (see FIGS. 10A and B). This was already found in previous studies, where liposomes stayed intact at low pH and leakage of macro molecules was not very pronounced [75, 101]. TELs are known to increase the stability of membranes at low pH, but the EPC/GCTE/Chol formulation showed the highest instability under those conditions and this effect was reduced by the addition of the two surfactant bio-enhancers TPGS and CS [102]. It seems likely, that the high rigidity of GCTE promotes membrane defects at high proton concentrations and that the fluidising effect by surfactants helps to reduce the leakage of small molecules [103, 104]. Aramaki et al. [105] found a CF release in pH 2 of around 60% for different EPC/Choi liposomes. However, the liposomes examined in Aramaki's study were multilamellar vesicles (MLV) in contrast to the extruded, unilamellar vesicles (ULV) used in the present study. MLV can be generally considered as more stable because the inner aqueous core of the liposomes is protected by multiple layers of membrane. The surprisingly high leakage of CF in this study might be also related to the method used. Before fluorescent measurement, the samples had to be neutralised, which led temporarily to different proton concentrations inside and outside the liposomes, assuming that the internal pH decreased during incubation time. At low pH inside the vesicles CF is non-ionic and thus better membrane-permeable, leading to a facilitated permeation of the marker to the outside, where the counter permeation is hindered due to the anionic charge of the fluorescent marker at neutral pH. This effect may also help to explain the somehow higher leakage for phospholipid-based vesicles found here compared to previous studies, where hydrophilic non-ionic marker like sucrose or polyvinylpyrrolidone was used [67, 77]. Still, the proton gradient can only play a role in CF leakage, when protons can diffuse into the inner aqueous compartment of the vesicles during the incubation in pH 2 buffer. Even if a macromolecule remains inside the liposomes, it is exposed to a high proton concentration, which could cause for example denaturation in terms of peptide drugs.

Human bile is described to contain predominantly the 5 bile salts chenodeoxycholic and cholic acid in equimolar concentration (40%), deoxycholic acid (15%) and lithocholic and ursodeoxycholic acid (together 5%). The bile salt concentration in the small intestine remains relatively constant at around 5-20 mM in duodenum and jejunum and decreases in the ileum due to reabsorption of the bile salts [106-108]. To facilitate the assay and to assure that the bile acid is fully dissolved, 10 mM sodium taurocholate was used in this study instead of the physiological mixture. This concentration is above the critical micelle concentration of sodium taurocholate. Thus, it should interact rapidly with the liposomes forming mixed vesicles and finally mixed micelles [109]. This might cause a total leakage of CF, which could indeed be observed for the EPC based liposomes (see FIG. 11C). However, the DLS data did not show any indication for the formation of micelles since size of vesicles was stable over the time and only the change in PI revealed some instability of the liposomes (see FIGS. 11A and B). FITC-dextran release of GCTE-free liposomes suggests either the transient formation of membrane pores large enough for the macromolecule to pass or the solubilisation of vesicles into mixed micelles and a continuous disturbance of membrane integrity (see FIG. 11D). Cholesterol is known to hamper forming of pores and membrane solubilisation, which is in good agreement with the low FITC-dextran release of EPC/Chol liposomes

In contrast, surfactants make the membrane more susceptible to sodium taurocholate. Also OT, which is not surface active, reduced the membrane stability, and this may be due to the lower amount of cholesterol in this formulation compared to EPC/Chol liposomes or the low melting point (24-31° C.) of OT. The stabilising effect of GCTE might be related both to its membrane spanning structure increasing the intermolecular forces in the membrane and to its rigidity hindering the insertion of sodium taurocholate into the membrane. Altogether, the stability results in bile salts suggest, that GCTE-stabilised liposomes remain with their membrane integrity and are likely to protect encapsulated drugs from degradation.

Ether lipids are known to be less susceptible against pancreatin, moreover Burns et al. [110] described a competitive inhibition of phospholipase A2 by diether lipids [77]. Taira et al. [75] found a CF release from conventional EPC/Chol liposomes in pancreatin of around 10% after 90 min, which is in good agreement with our results. The somewhat higher leakage of the EPC/GCTE/Chol formulation might be related not only to an enzymatic degradation of the lipids but also to protein/membrane interaction causing a destabilisation of the liposomal membrane (see FIG. 12C). The failure to improve stability against pancreatin of the TEL could be due to the fact that the amount of ester phospholipids is still 36% in the formulations giving phospholipase A2 enough targets. The stabilising effect of TPGS on the liposomes is probably due to sterical hinderance of the pancreatic enzymes by the PEG chain. Considering the size stability and the very low FITCdextran release the CF release in pancreatin is likely caused by small membrane defects and not by a rupture or total disintegration of some vesicles. Some of the effects could also be associated to membrane/protein interactions and not to enzymatic processes.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

### REFERENCES

[1] J. Hammen, G. Enslin, A. Kotze. Oral delivery of peptide drugs: barriers and developments, BioDrugs 19 (3) (2006) 165-177.
[2] G. Fricker, J. Drewe, Current concepts in intestinal peptide absorption, J. Pept. Sci. 2 (4) (1996) 195-211.
[3] E. Lipinski, F. Lombardo, B. Dominy, P. Feeney, Experimental and computational approaches to estimate solubility and permeability in drug discovery and development settings, Adv. Drug Deliv. Rev. 23 (1-3) (1997) 325.
[4] E. Ziv, M. Bendayan, Intestinal absorption of peptides through the enterocytes. Microsc. Res. Technol. 49 (4) (2000) 346-352.
[5] B. R. Stoll, H. R. Leipold, S. Milstein, D. A. Edwards. A mechanistic analysis of carrier-mediated oral delivery of protein therapeutics, J. Control. Release 64(1-3) (2000) 217-228.
[6] D. Malkov, R. Angelo, H.-Z. Wang, E. Flanders, H. Tang, I. Gomez-Orellana. Oral delivery of insulin with the eligen technology: mechanistic studies, Curr. Drug Del. 2(2) (2005) 191-197.
[7] A. des Rieux, V. Fievez, M. Garinot, Y.-J. Schneider, V. Preat, Nanoparticles as potential oral delivery systems of proteins and vaccines: a mechanistic approach, J. Control. Release 116 (1) (2006) 1-27.
[8] H. M. Patel, R. W. Stevenson, J. A. Parsons, B. E. Ryman, Use of liposomes to aid intestinal absorption of entrapped insulin in normal and diabetic docs, Biochim. Biophys. Acta 716 (2) (1982) 188-193.
[9] C. Chiang, N. Weiner, Gastrointestinal uptake of liposomes. II: in vivo studies, Int. J. Pharm. 40 (1-2) (1987) 143-150.
[10] A. Jesorka, O. Orwar, Liposomes: technologies and analytical applications, Annu. Rev. Anal. Chem. (1) (2008) 801-832.
[11] E. F. S. Authority, D-alpha-tocopheryl polyethylene glycol 1000 succinate (tpgs) in use for food for particular nutritional purposes. EFSA J. 490 (2007) 1-20.
[12] K. Iwanaga, S. Ono, K. Narioka, M. Kakemi, K. Morimoto, S. Yamashita, Y. Namba, N. Oku, Application of surface-coated liposomes for oral delivery of peptide: effects of coating the liposome's surface on the gi transit of insulin, J. Pharm. Sci. 88(2) (1999) 248-252.
[13] J. Ullienau, C. Schteingart. A. Hofmann, Physicochemical and physiological properties of cholylsarcosine—a potential replacement detergent for bile acid deficiency states in the small intestine, J. Clin. Invest. 89 (2) (1992) 420-431.
[14] S. Longmire-Cook, J. Lillienau, Y. Kim, C. Schteingart, R. Danzinger, O. Esch, A. Hofmann, Effect of replacement therapy with cholylsarcosine on fat malabsorption associated with severe bile acid malabsorption, Digest. Dis. Sci. 37 (8) (1992) 1217-1227.
[15] S. Eusufzai, Bile-acid malabsorption-mechanisms and treatment, Digest. Dis. 13(5) (1995) 312-321.
[16] K. Song, S. Chung, C. Shim, Enhanced intestinal absorption of salmon calcitonin (sct) from proliposomes containing bile salts. J. Control. Release 106(3) (2005) 298-308.
[17] Z. Degim, N. Unal, D. Essiz, U. Abbasoglu, The effect of various liposome formulations on insulin penetration across caco-2 cell monolayer, Life Sci. 75(23) (2004) 2819-2827.
[18] H. Natsume, S. Iwata, K. Ohtake, M. Miyamoto, M. Yamaguchi, K. Hosoya, D. Kobayashi, K. Sugibayashi, Y. Morimoto, Screening of cationic compounds as an absorption enhancer for nasal drug delivery, Int. J. Pharm. 185 (1) (1999) 112.
[19] T. Phoeung, L. M. Huber, M. Lafleur, Cationic detergent/sterol mixtures can form fluid lamellar phases and stable unilamellar vesicles, Langmuir 25 (10) (2009) 5778-5784.
[20] T. Schmitz, J. Hombach, A. Bernkop-Schnuerch, Chitosan-n-acetyl cysteine conjugates: In vitro evaluation of permeation enhancing and p-glycoprotein inhibiting properties. Drug Deliv. 15 (4) (2008) 245-252.
[21] M. Hayashi, M. Tomita, Mechanistic analysis for drug permeation through intestinal membrane, Drug Metab. Pharmacokinet. 22 (2) (2007) 67-77.
[22] A. D. Bangham, M. M. Standish, J. C. Watkins, Diffusion of univalent ions across the lamellae of swollen phospholipids, J. Mol. Biol. 13 (1) (1965) 238-252.
[23] J. Wegener, D. Abrams, W. Willenbrink, H. Galla, A. Janshoff, Automated multiwall device to measure transepithelial electrical resistances under physiological conditions, Biotechniques 37 (4) (2004) 590-597.
[24] D. Hunter, B. Frisken, Effect of extrusion pressure and lipid properties on the size and polydispersity of lipid vesicles, Biophys. J. 74 (6) (1998) 2996-3002.
[25] K. Yamabe, Potentiality of double liposomes containing salmon calcitonin as an oral dosage form, J. Control. Release 89 (3) (2003) 429-436.
[26] M. Webb, J. Wheeler, M. Bally, L. Mayer, The cationic lipid stearylamine reduces the permeability of the cationic drugs verapamil and prochlorperazine to lipid bilayers: implications for drug delivery, Bba-Biomembranes 1238 (2) (1995) 147-155.
[27] A. Baneljee, J. Roychoudhury, N. Ali, Stearylamine-bearing cationic liposomes kill leishmania parasites through surface exposed negatively charged phosphatidylserine, J. Antimicrob. Chemoth. 61(1) (2008) 103-110.
[28] F. Riddell, S. Arumugarn, Surface charge effects upon membrane transport processes: the effects of surface charge on the monensin-mediated transport of lithium ions through phospholipid bilayers studied by 7Li-NMR spectroscopy, Biochim. Biophys. Acta 945 (1) (1988) 65-72.
[29] A. Hildebrand, K. Beyer, R. Neubert, P. Garidel, A. Blume, Solubilization of negatively charged dppc/dppg liposomes by bile salts, J. Colloid Interface Sci. 279 (2) (2004) 559-571.
[30] T. Asakawa, H. Kitano, A. Ohta, S. Miyagishi, Convenient estimation for counterion dissociation of cationic micelles using chloride-sensitive fluorescence probe, J. Colloid Interface Sci. 242 (2) (2001) 284-287.
[31] M. Sadoqi. C. A. Lau-Cam, S. H. Wu, Investigation of the micellar properties of the tocopheryl polyethylene glycol succinate surfactants tpgs 400 and tpgs 1000 by steady state fluorometry, J. Colloid Interface Sci. 333 (2) (2009) 585-589.
[32] P.-Y. Ho, T.-K. Yeh, H.-T. Yao, H.-L. Lin, H.-Y. Wu, Y.-K. Lo, Y.-W. Chang, T.-H. Chiang, S. H. W. Wu, Y.-S. Chao, C.-T. Chen, Enhanced oral bioavailability of paclitaxel by d-alpha-tocopheryl polyethylene glycol 400 succinate in mice, Int. J. Pharm. 359 (1-2) (2008) 174-181.
[33] 0. Zschronig, K. Arnold, W. Richter, S. Ohki, Dextran sulfate-dependent fusion of liposomes containing cationic stearylamine, Chem. Phys. Lipids 63(1-2) (1992) 15-22.
[34] E. Swenson, W. Curatolo, Intestinal permeability enhancement for proteins, peptides and other polar drugs: mechanisms and potential toxicity, Adv. Drug Deliv. Rev. 8 (1) (1992) 39-92.
[35] E. J. van Hoogdalem. A. G. de Boer, D. O. Breimer, Intestinal drug absorption enhancement: an overview, Pharmacol. Ther. 44 (3) (1989) 407-443.
[36] K. Whitehead, N. Karr, S. Mitragotri, Safe and effective permeation enhancers for oral drug delivery, Pharm. Res. 25 (8) (2008) 1782-1788.
[37] S. Burgalassi, P. Chetoni, D. Monti, M. F. Saettone, Cytotoxicity of potential ocular permeation enhancers evaluated on rabbit and human corneal epithelial cell lines, Toxicol. Lett. 122 (1) (2001) 1-8.
[38] G. Mueller, A. Kramer, Biocompatibility index of antiseptic agents by parallel assessment of antimicrobial activity and cellular cytotoxicity, J. Antimicrob. Chemoth. 61(6) (2008) 1281-1287.
[39] Handbook of pharmaceutical excipients, fifth ed., American Pharmaceutical Association, Pharmaceutical Press, London, 2006 (p. 918).
[40] W. E. MacDonald, W. B. Deichmann, J. L. Radomski. B S. Austin, The chronic toxicity of octadecylamine in the rat—a supplemental report, Toxicol. Appl. Pharmacol. 4 (1962) 610-612.
[41] Y. Aramaki, S. Takano, H. Arima, S. Tsuchiya, Induction of apoptosis in wehi 231 cells by cationic liposomes, Pharm. Res. 17 (5) (2000) 515-520.
[42] E. Mayhew. M. Ito, R. Lazo, Toxicity of non-drug-containing liposomes for cultured human cells, Exp. Cell. Res. 171(1) (1987) 195-202.
[43] E. Collnot, C. Baldes, M. Wempe, J. Hyatt, L. Navarro, K. Edgar, U. Schaefer, C. Lehr, Influence of vitamin E tpgs poly(ethylene glycol) chain length on apical efflux transporters in caco-2 cell monolayers, J. Control. Release 111(1-2) (2006) 35-40.
[44] W. Krasavage, C. Terhaar, D-alpha-tocopheryl poly (ethylene glycol) 1000 succinate: acute toxicity, subchronic feeding, reproduction, and teratologic studies in the rat, J. Agric. Food Chem. 25 (2) (1977) 273-278.
[45] S. Michael, M. Thole, R. Dillmann, A. Fahr, J. Drewe, G. Fricker, Improvement of intestinal peptide absorption by a synthetic bile acid derivative cholylsarcosine, Ear. J. Pharm. Sci. 10 (2) (2000) 133-140.
[46] C. Kapral, F. Wewalka, V. Praxmarer, K. Lenz, A. Hofmann, Conjugated bile acid replacement therapy in short bowel syndrome patients with a residual colon, Z. Gastroenterol. 42 (07) (2004) 583-589.
[47] T. Furst, C. Bott, J. Stein, J. Dressman, Enteric-coated cholylsarcosine microgranules for the treatment of short bowel syndrome, J. Pharm. Pharmacol. 57 (1) (2005) 53-60.
[48] A. Schmassmann, M. Angellotto, H. Tonnu, C. Schteingart, S. Marcus. S. Rossi, A. Hofmann, Transport, metabolism, and effect of chronic feeding of cholylsarcosine, a conjugated bile acid resistant to deconjugation and dehydroxylation, Gastroenterology 98 (1) (1990) 163-174.
[49] B. J. Aungst. Intestinal permeation enhancers, J. Pharm. Sci. 89 (4) (2000) 429442.
[50] M. Hayashi, M. Tomita, S. Awazu, Transcellular and paracellular contribution to transport processes in the colorectal route, Adv. Drug Deliv. Rev. 28 (2) (1997) 191-204.
[51] T. Lindmark, Y. Kimura, P. Artursson, Absorption enhancement through intracellular regulation of tight junction permeability by medium chain fatty acids in caco-2 cells, J. Pharmacol. Exp.Ther.
[52] T. Shimazaki, M. Tomita, S. Sadahiro, M. Hayashi, S. Awazu, Absorption enhancing effects of sodium caprate and palmitoyl carnitine in rat and human colons, Digest. Dis. Sci. 43 (3) (1998) 641-645.
[53] M. Balda, J. Whitney, C. Flores, S. Gonzalez, M. Cereijido, K. Matter, Functional dissociation of paracellular permeability and transepithelial electrical resistance and disruption of the apical-basolateral intramembrane diffusion barrier by expression of a mutant tight junction membrane protein, J. Cell Biol. 134 (4) (1996) 1031-1049.
[54] G. Bathori, L Cervenak, I. Karadi, Caveolae—an alternative endocytotic pathway for targeted drug delivery, Crit. Rev. Ther. Drug 21(2) (2004) 67-95.
[55] T. Harder, K. Simons, Caveolae, digs, and the dynamics of sphingolipid cholesterol microdomains, Curr. Opin. Cell. Biol. 9 (4) (1997) 534-542.
[56] U. S. Huth, R. Schubert, R. Peschka-Suss, Investigating the uptake and intracellular fate of ph-sensitive liposomes by flow cytometry and spectral bioimaging, J. Control. Release 110 (3) (2006) 490-504.
[57] R. Yumoto, H. Nishikawa, M. Okamoto, H. Katayama, J. Nagai, M. Takano, Clathrin-mediated endocytosis of tire-albumin in alveolar type II epithelial cell line rle-6tn, Am. J. Physiol. Lung. Cell. Mol. Physiol. 290 (5) (2006) L946-55.
[58] B. M. Johnson, W. N. Charman, C. Porter, An in vitro examination of the impact of polyethylene glycol 400, pluronic p85, and vitamin E d-alpha-tocopheryl polyethylene glycol 1000 succinate on p-glycoprotein efflux and enterocyte-based metabolism in excised rat intestine, AAPS Pharmsci. 4(4) (2002) E40.
[59] B. D. Rege, J. P. Y. Kao, J. E. Polli, Effects of nonionic surfactants on membrane transporters in caco-2 cell monolayers, Eur. J. Pharm. Sci. 16 (4-5) (2002) 237246.
[60] E.-M. Collnot, C. Baldes, M. F. Wempe, R. Kappl. J. Huttermann, J. A. Hyatt, K. J. Edgar, U. F. Schaefer, C.-M. Lehr, Mechanism of inhibition of p-glycoprotein mediated efflux by vitamin E tpgs: influence on atpase activity and membrane fluidity, Mol. Pharm. 4 (3) (2007) 465-474.
[61] B. Aungst, H. Saitoh, D. Burcham, S. Huang, Enhancement of the intestinal absorption of peptides and nonpeptides, J. Control. Release 41 (1996) 19-31.
[62] S. Burgalassi, P. Chetoni, L. Dini, M. Najarro, D. Monti, P. Morelli, M. F. Saettone, Effect of permeation enhancers on buccal absorption, Arzneimittel-Forsch. 56 (7) (2006) 561-567.
[63] P. Artursson, Cell cultures as models for drug absorption across the intestinal mucosa, Crit. Rev. Ther. Drug 8 (4) (1991) 305-330.
[64] M. Gumbleton, A. G. Abulrob, L. Campbell, Caveolae: an alternative membrane transport compartment, Pharm. Res. 17 (9) (2000) 1035-1048.
[65] Patel, H., Ryman, B. E., 1976. Oral administration of insulin by encapsulation within liposomes. FEBS Lett. 62, 60-63.
[66] Fricker, G., Kromp, T., Wendel, A., Blume, A., Zirkel, J., Rebmann, H., Setzer, C., Quinkert, R.-O., Martin, F., Muller-Goymann, C., 2010. Phospholipids and lipid based formulations in oral drug delivery. Pharm. Res.
[67] Rowland, R. N., Woodley, J. F., 1980. The stability of liposomes in vitro to pH, bile salts and pancreatic lipase. Biochim. Biophys. Acta 620, 400-409.
[68] Iwanaga, K., Ono, S., Narioka, K., Morimoto, K., Kakemi, M., Yamashita, S., Nango, M., Oku, N., 1997. Oral delivery of insulin by using surface coating liposomes improvement of stability of insulin in GI tract. Int. J. Pharm. 157, 73-80.
[69] Takeuchi, H., Yamamoto, H., Niwa, T., Hino, T., Kawashima, Y., 1994. Mucoadhesion of polymer-coated liposomes to rat intestine in vitro. Chem. Pharm. Bull. 42, 1954-1956.
[70] Filipović-Grcić, J., Skalko-Basnet, N., Jalsenjak, I., 2001. Mucoadhesive chitosan coated liposomes: characteristics and stability. J. Microencapsul. 18, 3-12.
[71] Werle, M., Hironaka, K., Takeuchi, H., Hoyer, H., 2009. Development and in vitro characterization of liposomes coated with thiolated poly(acrylic acid) for oral drug delivery. Drug Dev. Ind. Pharm. 35, 209-215.
[72] Werle, M., Takeuchi, H., 2009. Chitosan-aprotinin coated liposomes for oral peptide delivery: development, characterisation and in vivo evaluation. Int. J. Pharm. 370, 26-32.
[73] Muramatsu, K., Maitani, Y., Nagai, T., 1996. Dipalmitoylphosphatidylcholine liposomes with soybean-derived sterols and cholesterol as a carrier for the oral administration of insulin in rats. Biol. Pharm. Bull. 19, 1055-1058.
[74] Han, M., Watarai, S., Kobayashi, K., Yasuda, T., 1997. Application of liposomes for development of oral vaccines: study of in vitro stability of liposomes and antibody response to antigen associated with liposomes after oral immunization. J. Vet. Med. Sci. 59, 1109-1114.
[75] Taira, M., Chiaramoni, N., Pecuch, K., Alonso-Romanowski, S., 2004. Stability of liposomal formulations in physiological conditions for oral drug delivery. Drug Deliv. 11, 123-128.
[76] Langworthy, T. A., 1977. Long-chain diglycerol tetraethers from Thermoplasma acidophilum. Biochim. Biophys. Acta 487, 37-50.
[77] Choquet, C. G., Patel, G. B., Beveridge, T. J., Sprott, G. D., 1994. Stability of pressure extruded liposomes made from archaeobacterial ether lipids. Appl. Microbiol. Biotechnol. 42, 375-384.
[78] Fan, Q., Relini, A., Cassinadri, D., Gambacorta, A., Gliozzi, A., 1995. Stability against temperature and external agents of vesicles composed of archaeal bolaform lipids and egg PC. Biochim. Biophys. Acta 1240, 83-88.
[79] Komatsu, H., Chong, P. L., 1998. Lowpermeability of liposomal membranes composed of bipolar tetraether lipids from thermoacidophilic archaebacterium Sulfolobus acidocaldarius. Biochemistry 37, 107-115.
[80] Patel, G. B., Chen, W., 2005. Archaeosome immunostimulatory vaccine delivery system. Curr. Drug Deliv. 2, 407-421.
[81] Lo, S. L., Chang, E. L., 1990. Purification and characterization of a liposomal forming tetraether lipid fraction. Biochem. Biophys. Res. Commun. 167, 238-243.
[82] Schouten, S., Van Der Meer, M. T. J., Hopmans, E. C., Rijpstra, W. I. C., Reysenbach, A.-L., Ward, D. M., Sinninghe Damste, J. S., 2007. Archaeal and bacterial glycerol dialkyl glycerol tetraether lipids in Hot Springs of Yellowstone National Park. Appl. Environ. Microbiol. 73, 6181-6191.
[83] De Rosa, M., Gambacorta, A., Gliozzi, A., 1986. Structure, biosynthesis, and physicochemical properties of archaebacterial lipids. Microbiol. Rev. 50, 70-80.
[84] Gliozzi, A., Paoli, G., Pisani, D., Gliozzi, F., De Rosa, M., Gambacorta, A., 1986. Phase transitions of bipolar lipids of thermophilic archaebacteria. Biochim. Biophys. Acta 861, 420-428.
[85] Gambacorta, A., Gliozzi, A., Rosa, M., 1995. Archaeal lipids and their biotechnological applications. World J. Microbiol. Biotechnol. 11, 115-131.
[86] Lo, S. L., Montague, C. E., Chang, E. L., 1989. Purification of glycerol dialkyl nonitol tetraether from Sulfolobus acidocaldarius. J. Lipid Res. 30, 944-949.
[87] Hill, M., 1978. Epidemiology and etiology of colon cancer. In: Grundmann, E. (Ed.), Colon Cancer Cancer Campaign, vol. 2, pp. 115-127.
[88] Nair, P., Turjman, N., 1983. Role of bile acids and neutral sterols in familial cancer syndromes of the colon. Dis. Colon Rectum 26, 629-632.
[89] Kandell, R., Bernstein, C., 1991. Bile-salt acid induction of DNA damage in bacterial and mammalian-cells—implications for colon cancer. Nutr. Cancer 16, 227-238.
[90] Latta, R., Fiander, H., Ross, N., Simpson, C., Schneider, H., 1993. Toxicity of bile-acids to colon-cancer cell-lines. Cancer Lett. 70, 167-173.
[91] Emmett, M., Guirl, M. J., Santa Ana, C. A., Porter, J. L., Neimark, S., Hofmann, A. F., Fordtran, J. S., 2003. Conjugated bile acid replacement therapy reduces urinary oxalate excretion in short bowel syndrome. Am. J. Kidney Dis. 41, 230-237.
[92] Takeuchi, H., Yamamoto, H., Kawashima, Y., 2001. Mucoadhesive nanoparticulate systems for peptide drug delivery. Adv. Drug Deliv. Rev. 47, 39-54.
[93] Whitmore, D. A., Wheeler, K. P., 1979. The fate of liposomes in the rat small intestine [proceedings]. Biochem. Soc. Trans. 7, 929-931.
[94] Rowland, R. N., Woodley, J. F., 1981a. The uptake of distearoylphosphatidylcholine/cholesterol liposomes by rat intestinal sacs in vitro. Biochim. Biophys. Acta 673, 217-223.
[95] Rowland, R. N., Woodley, J. F., 1981b. Uptake of free and liposome-entrapped 125I labelled PVP by rat intestinal sacs in vitro: evidence for endocytosis. Biosci. Rep. 1, 399-406.
[96] Rowland, R. N., Woodley, J. F., 1981c. Uptake of free and liposome-entrapped insulin by rat intestinal sacs in vitro. Biosci. Rep. 1, 345-352.
[97] Takeuchi, H., Matsui, Y., Sugihara, H., Yamamoto, H., Kawashima, Y., 2005. Effectiveness of submicron-sized, chitosan-coated liposomes in oral administration of peptide drugs. Int. J. Pharm. 303, 160-170.
[98] Werle, M., Makhlof, A., Takeuchi, H., 2010. Carbopol-lectin conjugate coated liposomes for oral peptide delivery. Chem. Pharm. Bull. 58, 432-434.
[99] Maher, S., Brayden, D. J., Feighery, L., McClean, S., 2008. Cracking the junction: update on the progress of gastrointestinal absorption enhancement in the delivery of poorly absorbed drugs. Crit. Rev. Ther. Drug 25, 117-168.
[100] Schubert, R., Schmidt, K. H., 1988. Structural changes in vesicle membranes and mixed micelles of various lipid compositions after binding of different bile salts. Biochemistry 27, 8787-8794.
[101] Patel, G. B., Agnew, B. J., Deschatelets, L., Fleming, L. P., Sprott, G. D., 2000. In vitro assessment of archaeosome stability for developing oral delivery systems. Int. J. Pharm. 194, 39-49.
[102] Elferink, M. G., de Wit, J. G., Driessen, A. J., Konings, W. N., 1994. Stability and protonpermeability of liposomes composed of archaeal tetraether lipids. Biochim. Biophys. Acta 1193, 247-254.
[103] Cavagnetto, F., Relini, A., Mirghani, Z., Gliozzi, A., Bertoia, D., Gambacorta, A., 1992. Molecular packing parameters of bipolar lipids. Biochim. Biophys. Acta 1106, 273-281.
[104] Gabriel, J. L., Chong, P. L., 2000. Molecular modeling of archaebacterial bipolar tetraether lipid membranes. Chem. Phys. Lipids 105, 193-200.
[105] Aramaki, Y., Tomizawa, H., Hara, T., Yachi, K., Kikuchi, H., Tsuchiya, S., 1993. Stability of liposomes in vitro and their uptake by rat Peyer's patches following oral administration. Pharm. Res. 10, 1228-1231.
[106] Borgstrom, B., Dahlqvis, T. A., Lund, H. G., Sjoval, L. J., 1957. Studies of intestinal digestion and absorption in the human. J. Clin. Invest. 36, 1521-1536.
[107] Hofmann, A. F., 1989. Enterohepatic circulation of bile acids. In: Handbook of Physiology, Section 6: The Gastrointestinal System III, pp. 567-595.
[108] Kararli, T. T., 1995. Comparison of the gastrointestinal anatomy, physiology, and biochemistry of humans and commonly used laboratory animals. Biopharm. Drug Dispos. 16, 351-380.
[109] Walde, P., Sunamoto, J., O'Connor, C. J., 1987. The mechanism of liposomal damage by taurocholate. Biochim. Biophys. Acta 905, 30-38.
[110] Burns, R. A., Friedman, J. M., Roberts, M. F., 1981. Characterization of short-chain alkyl ether lecithin analogues: ¹³C NMR and phospholipase studies. Biochemistry 20, 5945-5950.

1. A liposomal composition comprising:
a. a phospholipid;
b. cholesterol;
c. a permeability enhancer; and
d. an active pharmaceutical ingredient.

2. The composition as recited in claim 1 wherein said phospholipid is egg phosphatidylcholine.

3. The composition as recited in claim 2 wherein said permeability enhancer is selected from the group consisting of D-α-tocopheryl polyethylene glycol succinate, cholylsarcosine, cetylpyridinium chloride, and stearylamine.

4. The composition as recited in claim 3 wherein said permeability enhancer is D-α-tocopheryl polyethylene glycol 1000 succinate.

5. The composition as recited in claim 3 wherein said permeability enhancer is D-α-tocopheryl polyethylene glycol 400 succinate.

6. The composition as recited in claim 3 wherein said permeability enhancer is cholylsarcosine.

7. The composition as recited in claim 3 wherein said permeability enhancer is cetylpyridinium chloride.

8. The composition as recited in claim 3 wherein said permeability enhancer is stearylamine.

9. The composition as recited in claim 3 comprising an aqueous solution of said active pharmaceutical ingredient and a liposome-forming mixture comprising:
a. about 40% to about 60% of said phospholipid;
b. about 1% to about 30% of said permeation enhancers; and
c. about 10% to about 59% of said cholesterol.

10. The composition as recited in claim 9 comprising an aqueous solution of said active pharmaceutical ingredient and a liposome-forming mixture comprising:
a. about 50% of egg phosphatidylcholine;
b. about 5% to about 25% of said permeation enhancer; and
c. about 25% to about 45% of said cholesterol.

11. The composition as recited in claim 10 comprising an aqueous solution of said active pharmaceutical ingredient and a liposome-forming mixture comprising:
a. 50% of egg phosphatidylcholine;
b. 5% of D-α-tocopheryl polyethylene glycol 1000 succinate; and
c. 45% of cholesterol.

12. The composition as recited in claim 10 comprising an aqueous solution of said active pharmaceutical ingredient and a liposome-forming mixture comprising:
a. 50% of egg phosphatidylcholine;
b. 5% of D-α-tocopheryl polyethylene glycol 400 succinate; and
c. 45% of cholesterol.

13. The composition as recited in claim 10 comprising an aqueous solution of said active pharmaceutical ingredient and a liposome-forming mixture comprising:
a. 50% of egg phosphatidylcholine;
b. 10% of cholylsarcosine; and
c. 40% of cholesterol.

14. The composition as recited in claim 10 comprising an aqueous solution of said active pharmaceutical ingredient and a liposome-forming mixture comprising:
a. 50% of egg phosphatidylcholine;
b. 10% of cholylsarcosine;
c. 2.5% of stearylamine; and
d. 37.5% of cholesterol.

15. The composition as recited in claim 10 comprising an aqueous solution of said active pharmaceutical ingredient and a liposome-forming mixture comprising:
a. 50% of egg phosphatidylcholine;
b. 10% of stearylamine; and
c. 40% of cholesterol.

16. The composition as recited in claim 10 comprising an aqueous solution of said active pharmaceutical ingredient and a liposome-forming mixture comprising:
a. 50% of egg phosphatidylcholine;
b. 10% of cetylpyridinium chloride; and
c. 40% of cholesterol.

17. The composition as recited in claim 10 comprising an aqueous solution of said active pharmaceutical ingredient and a liposome-forming mixture comprising:
a. 50% of egg phosphatidylcholine;
b. 25% of cetylpyridinium chloride; and
c. 25% of cholesterol.

18. The composition as recited in claim 10, wherein said liposomes have an average diameter of 100 to 200 nm.

19. The composition as recited in claim 10, wherein said liposomes have a polydispersity index of 0.05 to 0.20.

20. A method of treatment of a disease comprising the administration of a therapeutically effective amount of a composition as recited in claim 1 to a patient in need thereof.

21. A method of treatment of a disease comprising the administration of a therapeutically effective amount of a composition as recited in claim 10 to a patient in need thereof.

22. A method of enhancing the permeation of an active pharmaceutical ingredient comprising the administration of a composition as recited in claim 1 to a patient.

23. The method of claim 22 wherein said permeation is increased by greater than 3-fold.

24. A method of enhancing the permeation of an active pharmaceutical ingredient comprising the administration of a composition as recited in claim 10 to a patient.

25. The method of claim 24 wherein said permeation is increased by greater than 3-fold.

26. A liposomal composition comprising:
a. a phospholipid;
b. cholesterol;
c. a purified glycerylcaldityl tetraether;
d. a permeability enhancer; and
e. an active pharmaceutical ingredient.

27. The composition as recited in claim 26 wherein said phospholipid is egg phosphatidylcholine.

28. The composition as recited in claim 26 wherein said permeability enhancer is selected from the group consisting of cholylsarcosine, octadecanethiol, and D-α-tocopheryl polyethylene glycol 1000 succinate.

29. The composition as recited in claim 26 wherein said composition does not contain any further tetraether lipid.

30. The composition as recited in claim 26 comprising an aqueous solution of said active pharmaceutical ingredient and a liposome-forming mixture comprising:
a. about 25% to about 80% of said phospholipid;
b. up to about 60% of said cholesterol;
c. about 5% to about 30% of said purified glycerylcaldityl tetraether; and
d. about 1% to about 35% of said permeability enhancers.

31. The composition as recited in claim 30 comprising an aqueous solution of said active pharmaceutical ingredient and a liposome-forming mixture comprising:
a. about 36% of said phospholipid;
b. about 40% to about 54% of said cholesterol;
c. about 9% of said purified glycerylcaldityl tetraether; and
d. about 1% to about 15% of said permeability enhancers.

32. The composition as recited in claim 26 comprising an aqueous solution of said active pharmaceutical ingredient and a liposome-forming mixture comprising:
a. about 25% to about 80% of egg phosphatidylcholine;
b. up to about 60% of cholesterol;
c. about 5% to about 30% of purified glycerylcaldityl tetraether; and
d. about 1% to about 35% of a permeability enhancer, selected from the group consisting of cholylsarcosine, octadecanethiol, and D-α-tocopheryl polyethylene glycol 100 succinate.

33. The composition as recited in claim 30 comprising an aqueous solution of said active pharmaceutical ingredient and a liposome-forming mixture comprising:
a. about 36% of egg phosphatidylcholine;
b. about 40% to about 54% of cholesterol;
c. about 9% of purified glycerylcaldityl tetraether; and
d. about 1% to about 15% of a permeability enhancer, selected from the group consisting of cholylsarcosine, octadecanethiol, and D-α-tocopheryl polyethylene glycol 100 succinate.

34. The composition as recited in claim 26, wherein said liposomes have an average diameter of 100 to 200 nm.

35. The composition as recited in claim 26, wherein said liposomes have a polydispersity index of 0.05 to 0.20.

36. A method of treatment of a disease comprising the administration of a therapeutically effective amount of a composition as recited in claim 26 to a patient in need thereof.

37. A method of treatment of a disease comprising the administration of a therapeutically effective amount of a composition as recited in claim 32 to a patient in need thereof.

38. A method of enhancing the permeation of an active pharmaceutical ingredient comprising the administration of a composition as recited in claim 26 to a patient.

39. The method of claim 38 wherein said permeation is increased by greater than 3-fold.

40. A method of enhancing the permeation of an active pharmaceutical ingredient comprising the administration of a composition as recited in claim 32 to a patient.

41. The method of claim 40 wherein said permeation is increased by greater than 3-fold.
